(19) United States
(12) Patent Application Publication
(10) Pub. No.: US 2024/0091067 A1
(43) Pub. Date: Mar. 21, 2024

(54) OPHTHALMIC MEDICAL DEVICE FOR TREATMENT OF MEIBOMIAN GLAND DYSFUNCTION AND EVAPORATIVE DRY EYE DISEASE UTILIZING SHEAR THINNING TECHNOLOGY

(71) Applicant: EYEDETEC MEDICAL, INC., Danville, CA (US)

(72) Inventors: Barry J. LINDER, Scottsdale, AZ (US); Ronald D. LINDER, Gilbert, AZ (US); James L. DIPPO, Arvada, CO (US); Rubin D. LINDER, Scottsdale, AZ (US); John A. SCHOLL, San Ramon, CA (US); Vance THOMPSON, SIOUX Falls, SD (US)

(73) Assignee: EYEDETEC MEDICAL, INC., Danville, CA (US)

(21) Appl. No.: 18/522,857

(22) Filed: Nov. 29, 2023

Related U.S. Application Data
(63) Continuation-in-part of application No. 16/739,958, filed on Jan. 10, 2020.
Application No. 16/739,958 is a continuation-in-part of application No. 16/244,820, filed on Jan. 10, 2019.
Application No. 16/244,820 is a continuation-in-part of application No. 16/146,396, filed on Sep. 28, 2018.
Application No. 16/146,396 is a continuation-in-part of application No. 15/187,457, filed on Jun. 20, 2016.
(60) Provisional application No. 62/615,741, filed on Jan. 10, 2018.
(60) Provisional application No. 62/565,818, filed on Sep. 29, 2017.
(60) Provisional application No. 62/230,843, filed on Jun. 18, 2015.

(51) Int. Cl. A61F 9/04; A61F 7/00; A61F 7/08

(52) U.S. Cl. CPC A61F 9/04; A61F 7/007; A61F 7/08; A61F 2007/0004

(57) ABSTRACT

Embodiments provide a moldable warming device is configured to provide, using a heating disc, therapeutic warmth and, using a harmonic RFSVG, tuned harmonic resonance frequency stimulation vibration according to determined tuning parameters across the contour of the entire surface of the user's eyelid and periorbital structures to induce expression of shear thinned and liquified meibum from each Meibomian gland in all four eyelids to improve the lipid layer of the surface of the user's eye tear film. According to an embodiment, the moldable warming device further includes a pair of removable and reusable eye pads, each eye pad including a miniaturized optical coherence tomography device configured to map, in real-time, a topography of the user's periocular surface, including a mapping of the eyes and the nose including interpupillary distance and corneal apex height above the lateral canthus to optimize conformance of the pair of eye pads to the periocular surface of the user's eyelids.

### CROSS REFERENCE TO RELATED APPLICATIONS

This application is a continuation-in-part application of U.S. patent application Ser. No. 16/739,958, filed on Jan. 10, 2020; U.S. patent application Ser. No. 16,739,958 is a continuation-in-part application of U.S. patent application Ser. No. 16/244,820, filed on Jan. 10, 2019, which claims priority to U.S. Provisional Patent Application Ser. No. 62/615,741, filed on Jan. 10, 2018; U.S. patent application Ser. No. 16,244,820 is a continuation-in-part application of U.S. patent application Ser. No. 16/146,396, filed on Sep. 28, 2018, which claims priority to U.S. Provisional Patent Application Ser. No. 62/565,818, filed on Sep. 29, 2017; U.S. patent application Ser. No. 16/146,396 is a continuation-in-part application of U.S. patent application Ser. No. 15/187,457, filed on Jun. 20, 2016, which claims priority to U.S. Provisional Patent Application Ser. No. 62/230,843, filed on Jun. 18, 2015, all of the above-referenced applications are hereby incorporated by reference in their entireties into this application.

### BACKGROUND

### Field

Embodiments relate generally to devices and methods for treating and monitoring dry eye disease. More particularly, embodiments of the disclosure relate to wearable eye masks that include heating elements and resonance frequency vibration stimulation generators for providing liquefaction and mobilization of meibum within the eyelid's Meibomian glands and for activating the lacrimal functional unit to effect neuromodulation of additional tear production including shear-thinning for improved Meibomian gland expression.

### Description of the Related Art

Eye patch devices are known in the art. For example, U.S. Pat. No. 4,682,371 (“the '371 patent”) describes a protective eye patch. The patch described in the '371 patent includes several tabs for securing the patch to a patient's eye. U.S. Pat. No. 3,068,863 describes a patch designed to keep the eye closed. U.S. Pat. No. 3,092,103 describes a patch with a cushion material at the edge that allows the patient's eye to move underneath the eye patch. U.S. Pat. No. 3,908,645 describes an ophthalmic therapeutic pressure bandage with a conformable, permeable carrier tape.

U.S. Pat. No. 6,409,746 describes an eye pillow that releases steam from its surface applied to the eyes and the area around the eyes. The temperature described in the '746 patent is 50° C. or lower and has a total weight of 50 g or more.

Several conditions exist for which medical and cosmetic therapy is appropriate. For example, blepharitis, meibomitis, chalazia, and/or styes are common disorders of the eyelids that cause chronic inflammation in the peri-orbita and are often associated with ocular tear film abnormalities resulting in dry eye disease and symptoms. Symptoms of dry eye disease and blepharitis include burning, itching, light sensitivity, blurred vision, tearing, and foreign body sensation. Signs include eyelash crusting, ocular discharge, eyelid scaling and swelling, corneal staining, and conjunctival redness. For example, staphylcoccal blepharatis is often associated with scaling and crusting along the eye lashes. There is no cure for dry eyes, and long-term treatment is required to keep it under control.

The predominant cause of dry eye is an insufficient or abnormal lipid layer of the surface of the tear film. In a healthy eye, this oily layer inhibits the evaporation of the water-based sub layers of the tear film, thereby maintaining a stable tear film. These lipids are produced in the Meibomian glands located in the eyelids. From about 24 to about 40 Meibomian glands exist in each eyelid. For those suffering evaporative dry eye disease, the likely root cause is Meibomian glands that have become filled with viscous lipids, and occasionally clogged, resulting in a reduced quantity and abnormal quality of lipids flowing out onto the tear film. Meibomitis, also known as Meibomian Gland Dysfunction (MGD), is a dysfunction of the Meibomian gland and limits the gland's ability to provide a normal lipid-based oily layer as a critical component of the eye's natural tear film.

Currently available treatments for dry eye disease and related conditions include warm compresses for 5-15 minutes, such as a warm washcloth or an Eyedetec Medical EyeGiene® Insta-Warmth heater, that heats the debris and crust on the lid, and lowers the viscosity of the lipids in the Meibomian glands. After the lid has been warmed, occasionally a lid scrub is performed by using a suitable soap, such as Neutrogena® or Johnson's Baby Shampoo®. Commercially available cleansing pads are available to assist in performing the lid scrub, for example OCuSOFT® Lid Scrubs or Novartis Ophthalmics Eye Scrub®. Following the eye scrub, antibiotics, such as polysporin, tobramycin, or erythromycin, can be applied to alleviate patient discomfort and reinforce the treatment.

Warm compress therapy applied to the skin of the closed eyelids increases tear-film lipid layer thickness for subjects with MGD by more than 80% after 5 minutes of initiating treatment and an additional 20% after 15 minutes of treatment. The transition temperature from a solid to a liquid for Meibomian lipids is a range from 28° C. to 32° C. because of differences between an individual's mixture of lipids. The temperature of the eyelids will therefore affect the liquidity of Meibomian lipids and hence their viscosity. The non-Newtonian lipid mixture is known to undergo shear thinning when exposed to shear forces. Further it is known that oscillations enhance the flow rate of a shear-thinning fluid.

Conventional ocular heating devices, such as warm compresses, typically require an external power source. These sources include electricity, a stove top boiling preparation, or a microwave appliance, and are consequently difficult to provide a controlled temperature to the eyelids, are labor intensive, cumbersome, and inconvenient, and therefore historically result in poor patient compliance and persistence with the recommended therapy. Some success is realized with in-office, doctor-assisted visits.

What is needed is a convenient, accurate, and effective, easily used hand moldable heating source that patients or their doctors apply via a coupling mechanism to a patient's eyelids, and which delivers a therapeutic temperature to the entire eyelid surface independent of the individual's orbital anatomy, for a sufficient length of time to be effective. There is also a need for a device or component of the system that incorporates a moldable material to serve as a coupling element, able to sequentially and alternately deliver heat and resonance frequency vibration stimulation to the target tissue across the entire surface of the user's eyelid and periorbital structures, as well as to optionally detect a positive eyelid resonant response from a broad range of generated harmonic frequencies, thus allowing a personalized or custom approach to each individual user. There is also a need for a device or component of the system incorporating a neurostimulation unit to deliver resonance or non-resonance frequency vibration stimulation to the sensory nerves in the nasal area to induce neuromodulation of the tear production reflex including Meibomian gland expression.

### SUMMARY

Embodiments provide a moldable warming device is configured to sequentially and alternately apply, using a heating disc, therapeutic warmth and, using a harmonic resonance frequency stimulation vibration generator (RFSVG), tuned harmonic resonance frequency stimulation vibration according to determined tuning parameters across the contour of the entire surface of the user's eyelid and periorbital structures to induce expression of shear thinned and liquified meibum from each Meibomian gland in all four eyelids to improve the lipid layer of the surface of the user's eye tear film. According to an embodiment, the moldable warming device further includes a pair of removable and reusable eye pads, each eye pad including a miniaturized optical coherence tomography device configured to map, in real-time, a topography of the user's periocular surface, including a mapping of the eyes and the nose including interpupillary distance and corneal apex height above the lateral canthus to optimize conformance of the pair of eye pads to the periocular surface of the user's eyelids.

In particular, according to an embodiment, there is provided a moldable warming device. The moldable warming device includes a heating disc, a harmonic RFSVG, a coupling device, a mask configured to hold the heating disc, the harmonic RFSVG, and the coupling device for use in parallel utility, and a sensor configured to determine tuning parameters of a harmonic resonance frequency stimulation vibration and heating profile of a user's individual eyelid and periorbital three-dimensional anatomy and surface topography. The moldable warming device is configured to sequentially and alternately apply, using the heating disc, therapeutic warmth and, using the harmonic RFSVG, tuned harmonic resonance frequency stimulation vibration according to the determined tuning parameters across the contour of the entire surface of the user's eyelid and periorbital structures to induce expression of shear thinned and liquified meibum from each Meibomian gland in all four eyelids to improve the lipid layer of the surface of the user's eye tear film. The RFSVG is configured to generate the tuned harmonic resonance frequency stimulation vibration comprising a superposition of two or more longitudinal vibrational waves across the contour of the entire surface of the user's eyelid and periorbital structures, wherein a primary axis of the first vibrational wave is in a superior-inferior direction parallel to the depth of the user's Meibomian glands, and a primary axis of the second vibrational wave is in a medial-lateral direction tangential to the depth of the user's Meibomian glands, to induce expression of the shear thinned and liquified meibum from each Meibomian gland in all four eyelids to improve the lipid layer of the surface of the user's eye tear film.

According to an embodiment, the moldable warming device further includes a pair of removable and reusable eye pads. Each eye pad includes a miniaturized optical coherence tomography device configured to map, in real-time, a topography of the user's periocular surface, including a mapping of the eyes and the nose including interpupillary distance and corneal apex height above the lateral canthus, and based on the real-time mapping, determine a precise location for positioning each eye pad to quantify the position of each eye pad and to correlate the adjustment of the pair of eye pads together, so that both eye pads, in concert, expand or contract their lateral position initially based on the interpupillary distance.

According to an embodiment, the miniaturized optical coherence tomography device is further configured to quantify, based on the real-time mapping, the position for each eye pad, wherein each eye pad is configured in concert to move in and out in a lateral direction, based on a measurement of the corneal apex height relative to the lateral canthus.

According to an embodiment, the miniaturized optical coherence tomography device is further configured to determine, based on the real-time mapping, a surface topography and a periocular surface of the user's eyes to optimize conformance of the pair of eye pads to the periocular surface of the user's closed eyelids.

According to an embodiment, the moldable warming device further includes a plurality of manual adjustment wheels. Each manual adjustment wheel is configured to produce tactile feedback to the user of the moldable warming device allowing the user to manually adjust each eye pad to optimize conformance of the pair of eye pads to the periocular surface of the user's eyelids.

According to an embodiment, each eye pad includes a module insert configured to perform advanced diagnostic enabled imaging. The module insert includes one of: (1) a corneal staining pad, the corneal staining pad being one of a corneal fluorescein staining pad or a corneal rose bengal staining pad with illumination of selectable variable waverlengths, the corneal staining pad configured to detect dry eye syndrome, (2) a tear interferometer configured to measure a tear meniscus in the user's eyes, (3) a conjunctival redness measurement pad configured to diagnose and assess a level of conjunctival redness in the user's eyes, (4) a tear breakup time measurement pad configured to measure tear breakup time in the user's eyes, (5) a blink rate measurement pad configured to assess blink rate and completeness of blink cycle of the user's eyes for maintaining tear film stability and for sustaining tear film homeostasis, (6) an intraocular cell and flare measurement pad configured to measure intraocular cell and flare in the user's eyes, and (7) an intraocular pressure measurement pad configured to measure intraocular pressure in the user's eyes.

According to an embodiment, the pair of eye pads are configured to provide cold compress for inflammation reduction.

According to an embodiment, the pair of eye pads further include a surface available to hold hydrogel sheets or moisturized cotton pads. The hydrogel sheets or the moisturized cotton pads contain either moisture alone or impregnated with anti-inflammatory agents including steroids or non-steroidal anti-inflammatories or agents expanding therapies including blepharitis, demodex, or eyelid wrinkles.

According to another embodiment, there is provided a method for ophthalmic eyelid therapy. The method includes the steps of: applying a moldable warming device to a user's individual eyelid and periorbital three-dimensional anatomy and surface topography, determining, via a sensor, tuning parameters of a harmonic resonance frequency stimulation vibration and heating profile of the user's individual eyelid and periorbital three-dimensional anatomy and surface topography, sequentially and alternately applying, using a heating disc, thermal energy and, using a harmonic RFSVG, harmonic resonance frequency stimulation vibration according to the determined heating profile of the user's individual eyelid and periorbital three-dimensional anatomy and surface topography and the tuning parameters, and transferring the thermal energy and the harmonic resonance frequency stimulation vibration across the contour of the entire surface of the user's eyelid and periorbital structures, and to induce expression of shear thinned and liquified meibum from each Meibomian gland in all four eyelids to improve the lipid layer of the surface of the user's eye tear film.

According to an embodiment, the moldable warming device includes the heating disc, the harmonic RFSVG, a coupling device, a mask, where the mask is configured to hold the heating disc, the harmonic RFSVG, and the coupling device for use in parallel utility, and the sensor.

According to an embodiment, the harmonic resonance frequency stimulation vibration includes a superposition of two or more longitudinal vibrational waves across the contour of the entire surface of the user's eyelid and periorbital structures, where a primary axis of the first vibrational wave is in a superior-inferior direction parallel to the depth of the user's Meibomian glands, and a primary axis of the second vibrational wave is in a medial-lateral direction tangential to the depth of the user's Meibomian glands to induce expression of shear thinned and liquified meibum from each Meibomian gland in all four eyelids to improve the lipid layer of the surface of the user's eye tear film.

According to an embodiment, each eye pad includes a miniaturized optical coherence tomography device. The method for ophthalmic eyelid therapy further includes, using the miniaturized optical coherence tomography device, the steps of mapping, in real-time, a topography of the user's periocular surface, including a mapping of the eyes and the nose including interpupillary distance and corneal apex height above the lateral canthus, and based on the real-time mapping, determining a precise location for positioning each eye pad to quantify the position of each eye pad and to correlate the adjustment of the pair of eye pads together, so that both eye pads, in concert, expand or contract their lateral position initially based on the interpupillary distance.

According to an embodiment, the method for ophthalmic eyelid therapy further includes the step of quantifying, using the miniaturized optical coherence tomography device, based on the real-time mapping, the position for each eye pad, where each eye pad is configured in concert to move in and out in a lateral direction, based on a measurement of the corneal apex height relative to the lateral canthus.

According to an embodiment, the method for ophthalmic eyelid therapy further includes the step of determining, based on the real-time mapping, a surface topography and a periocular surface of the user's eyes to optimize conformance of the pair of eye pads to the periocular surface of the user's closed eyelids.

According to an embodiment, the moldable warming device further includes a plurality of manual adjustment wheels, the method for ophthalmic eyelid therapy further including the step of manually adjusting, using each manual adjustment wheel configured to produce tactile feedback to the user of the moldable warming device, each eye pad to optimize conformance of the pair of eye pads to the periocular surface of the user's eyelids.

According to an embodiment, each eye pad includes a module insert, the method for ophthalmic eyelid therapy further including performing, using the module insert, advanced diagnostic enabled imaging, where the performing step includes one of: (1) detecting, using a corneal staining pad, the corneal staining pad being one of a corneal fluorescein staining pad or a corneal rose bengal staining pad with illumination of selectable variable waverlengths, dry eye syndrome, (2) measuring, using a tear interferometer, a tear meniscus in the user's eyes, (3) diagnosing and assessing, using a conjunctival redness measurement pad, a level of conjunctival redness in the user's eyes, (4) measuring, using a tear breakup time measurement pad, tear breakup time in the user's eyes, (5) assessing, using a blink rate measurement pad, blink rate and completeness of blink cycle of the user's eyes for maintaining tear film stability and for sustaining tear film homeostasis, (6) measuring, using an intraocular cell and flare measurement pad, intraocular cell and flare in the user's eyes, and (7) measuring, using an intraocular pressure measurement pad, intraocular pressure in the user's eyes.

According to an embodiment, the pair of eye pads are configured to provide cold compress for inflammation reduction.

According to an embodiment, the pair of eye pads further include a surface available to hold hydrogel sheets or moisturized cotton pads, the hydrogel sheets or the moisturized cotton pads containing either moisture alone or impregnated with anti-inflammatory agents including steroids or non-steroidal anti-inflammatories or agents expanding therapies including blepharitis, demodex, or eyelid wrinkles.

### BRIEF DESCRIPTION OF THE DRAWINGS

So that the manner in which the recited features, aspects, and advantages of the disclosure, as well as others that will become apparent, are attained and can be understood in detail, a more particular description of certain embodiments briefly summarized above can be had by reference to the embodiments that are illustrated in the drawings that form a part of this specification. It is to be noted, however, that the appended drawings illustrate only exemplary embodiments and are, therefore, not to be considered limiting of the disclosure's scope, for the disclosure can admit to other equally effective embodiments. Like numbers refer to like elements throughout, and the prime notation, if used, indicates similar elements in alternative embodiments or positions.

FIG. 1A is a front view of a heating disc device; FIG. 1B is a side or section view of the heating disc.

FIGS. 2A-2F are graphs of the force velocity and differential pressure for various porous polyolefin compositions.

FIG. 3A is a front view of a packaged heating disc; FIG. 3B is a section view of a packaged heating disc.

FIG. 4A and FIG. 4B are a side section view and front section view heater disk showing embedded metallic nanoparticles.

FIG. 5A and FIG. 5B are a side section view and front section view heater disk showing embedded metallic and ceramic nanoparticles.

FIG. 6A is a front view of the heating disc with attached resonance frequency stimulation generator; FIG. 6B is a side view of the heating disc with attached resonance frequency stimulation generator and USB connector.

FIG. 7A is a reusable mask incorporating both a miniature resonance frequency stimulation vibration generator and permanent imbedded heater element; FIG. 7B shows an example vibration modulation controller; FIG. 7C shows an example mechanical resonator;

FIG. 7D shows an example control board.

FIG. 8 is a representative far infrared front-end spot heater.

FIG. 9A is a section view of a microfluidic sensor; FIG. 9B is a front view of a microfluidic sensor.

FIG. 10A is a schematic for a dual channel solution sensing electrode; FIG. 10B is a schematic for a single channel solution sensing electrode.

FIG. 11 is a photograph of the mask prototype with the proposed 3D shape memory element.

FIG. 12A is a front view of the EyeGiene® heating device demonstrating temperature and frequency stimulation generator possible mask locations, along with in-place signal conditioner and amplifying circuitry. FIG. 12B is a top view of the original moldable heater with the new addition of a miniature resonance frequency stimulation generator located in the new pocket which is formed when the moldable heater is manufactured. The frequency stimulation generator is a reusable electrical device, which snaps into place for the duration of the one-time use or reusable heater element and is then removed before the one-time use or reusable heater element is removed. This view shows the heat shield material incorporated into the design for thermal manipulation of heater element, energy output.

FIG. 13 further demonstrates the frequency stimulation controller with a temperature controller incorporated into the unit.

FIG. 14 is an electrical schematic demonstrating the circuitry to localize and transmit sensor information via antenna to a smartphone or smart device receiver, processor, and display.

FIG. 15 is a block diagram of the path to transmit data to a smartphone or smart device receiver, processor, and display.

FIG. 16 further shows a block diagram showing a smartphone or smart device as the receiver, processor, and display.

FIG. 17A is a front view drawing of a single location temperature sensor and frequency stimulation generator. The thermocouples have wireless transmission capability, and the power is provided by a localized power source, replaceable battery, or USB. FIG. 17B further shows a top view of the single point sensor showing the sensor on the eyelid side of the mask.

FIGS. 18A-18C are photographs depicting a single temperature sensor with wireless transmission capabilities.

FIG. 19 is a drawing of the front view of a multiple temperature sensor array using a flexible printed circuit for eye shape capabilities and data collection via wireless transmission to external components such as a smartphone or smart device, wireless receiver, USB, processor, or display.

FIGS. 20A and 20B show photographs of a flexible printed circuit with temperature sensors or sensor array.

FIG. 21 shows a mask with multiple sensor capability. The sensor array detects temperature, pressure, moisture, pH, and frequency stimulation. This array communicates via wireless technology to a smartphone or smart device.

FIG. 22A shows a photograph of a miniaturized temperature sensing device. FIG. 22B shows a performance graph of the miniaturized temperature sensing device. FIG. 22C shows a depiction of a miniaturized moisture/humidity sensing device. FIGS. 22D-1 and 22D-2 shows photographs of a miniaturized pressure sensing device. FIG. 22E shows a depiction of a miniaturized pH sensing device. FIG. 22F shows a photograph of the miniaturized pH sensing device.

FIG. 23 is a commercial wire schematic for a smartphone or smart device to receive process and display data from the wireless sensor array.

FIG. 24A is a graphical representation showing a front view of a human eye. FIG. 24B is a graphical representation showing an enlarged cross-sectional view of an upper eyelid of the human eye. FIG. 24C is a graphical representation showing a cross-sectional view through a Meibomian gland of an upper eyelid of a human eye where the harmonic resonance frequency stimulation vibration mobilizes and stimulates flow of the Meibum lipids within a Meibomian gland. The mobilized and stimulated flow leads to enhanced excretion of the Meibum lipids.

FIG. 25 is a front view drawing of a heating mask including a neurostimulation unit for providing resonance or non-resonance frequency stimulation vibration to the nasal sensory nerves for inducing through controlled neuromodulation a tear production reflex including Meibomian Gland expression.

FIGS. 26A-26C show a moldable warming device incorporating a facial mapping device in each of the replaceable, adjustable, moldable eye pads of the mask.

FIG. 27 shows a top view of a moldable warming device including a plurality of manual adjustment wheels, right and left eye pad adjustment wheels, configured to optimally adjust the positioning of the eye pads of the mask.

FIGS. 28A-28C show a moldable warming device incorporating a facial mapping device in each of the replaceable, adjustable, moldable eye pads of the mask.

FIGS. 29A-29C show a moldable warming device incorporating a corneal fluorescein staining pad in each of the replaceable, adjustable, moldable eye pads of the mask.

FIGS. 30A-30C show a moldable warming device incorporating a tear meniscus device in each of the replaceable, adjustable, moldable eye pads of the mask.

FIGS. 31A-31C show a moldable warming device having a conjunctival redness measurement device in each of the replaceable, adjustable, moldable eye pads of the mask.

FIGS. 32A-32C show a moldable warming device incorporating a tear breakup time measurement device in each of the replaceable, adjustable, moldable eye pads of the mask.

FIGS. 33A-33C show a moldable warming device incorporating a blink rate measurement device in each of the replaceable, adjustable, moldable eye pads of the mask.

FIGS. 34A-34C show a moldable warming device incorporating an intraocular cell and flare measurement device in each of the replaceable, adjustable, moldable eye pads of the mask.

FIGS. 35A-35C show a moldable warming device incorporating an intraocular pressure measurement device in each of the replaceable, adjustable, moldable eye pads of the mask.

### DETAILED DESCRIPTION

While the scope of the system and method will be described with several embodiments, it is understood that one of ordinary skill in the relevant art will appreciate that many examples, variations and alterations to the systems and methods described here are within the scope and spirit of the embodiments.

Accordingly, the embodiments described are set forth without any loss of generality, and without imposing limitations, on the embodiments. Those of skill in the art understand that the scope includes all possible combinations and uses of particular features described in the specification.

As used throughout, one of ordinary skill in the relevant art would understand that the following terms can all be used interchangeably: dry eyes, dry eye disease, dry eye syndrome, evaporative dry eye, lipid deficiency dry eyes, blepharitis, Meibomian gland disease, Meibomian gland dysfunction, and MGD.

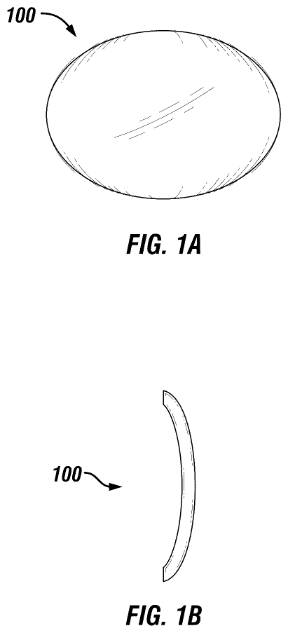

Referring now to the disclosed devices in more detail, in FIG. 1A and FIG. 1B, there is shown a heating disc 100 manufactured to fit comfortably in a reusable mask conforming to the natural shape of the closed eyelid surface and surrounding periorbital area. A suitable shape is an oval configuration better describing the shape of the ocular surface but can also be manufactured in rectangle or square shapes that are bigger than this minimum oval requirement.

In further detail, still referring to the devices disclosed in FIG. 1A and FIG. 1B, the heating disc 100 shows a side view of an initial curvature provided to conform the device to the closed eyelid surface by the patient when in use. The device is manufacturable such that it has a first configuration, such as an initial curvature as shown, and is conformable by the patient, physician or technician into a second configuration when in use. The material is pliable and can be easily shaped to maximize the contact area between the mask and the closed eyelid surface, to accurately match an individual user's particular orbital anatomy. For example, there will be a difference in shape for a user with a deeply set eye and deep orbit versus a user with an anteriorly placed globe. The heating disc 100 can be shaped by hand to fit a particular orbital anatomy. This approach will accurately match an individual user's particular orbital anatomy with the heating disc's high shape retention characteristics. Alternatively, light compression from the reusable eye mask will conform the heating disc 100 to the surface of the closed eyelids.

The desired heating disc integrity and heating duration are achieved by controlling disc thickness, formulation of the heating material, and porosity that allows controlled air flow to the heating material. Ideal time for application of heat is in a range from about 5 to 30 minutes, preferably from about 5 to 15 minutes and temperature at the surface of the eyelid should be between about 40 and 46° C. Example polyethylene (PE) based materials with a usable 25-60 μm pore size (PE25 through PE60) are shown in Table 1.

A nominal pore volume of 50% will allow the heating disc 100 to be reshaped or molded by the patient. The porous and moldable PE based material can have nominal pore sizes of 7-150 μm and are manufactured up to 300 μm in pore size. Another polyolefin material, polypropylene (PP), (PP-100 and PP150), shown in Table 1, is a heating disc material with 100-150 μm pore size with a smaller 45% pore volume, and can be infused with larger heater material particles for a longer disc heating time of 20-25 minutes.

TABLE 1
Porous plastic and moldable materials

| Material Parameter | PE10 | PE25 | PE60 | PE100 | PE125 | PE-HV | PP50 | PP100 | PP150 | PVDF30 | PTFE30 |
|---|---|---|---|---|---|---|---|---|---|---|---|
| Polymer Type | PE | PE | PE | PE | PE | PE | PP | PP | PP | PVDF | PTFE |
| Material Options | H2O/SS | H2O/SS | H2O/SS | H2O/SS | H2O/SS | H2O/SS | H2O/SS | H2O/SS | H2O/SS | | |
| Nominal Pore Size (μ) | 10 | 25 | 60 | 100 | 150 | 30 | 50 | 100 | 150 | 30 | 30 |
| Nominal Pore Volume (%) | 50 | 50 | 50 | 50 | 50 | 60 | 45 | 45 | 45 | 45 | 40 |
| Air Permeability | <10 | 10-50 | 30-90 | 50-100 | 70-120 | 40-80 | 40-120 | 60-150 | 70-200 | 10-50 | 5-25 |
| (ft/min @ 1.2″ H2O ΔP, | | | | | | | | | | | |
| material thickness of .125″) | | | | | | | | | | | |
| H2O Intrusion Pressure | 175 | 152 | 70 | 42 | 16 | 65 | 35 | 12 | 10 | 60 | 80 |
| (mBar) | | | | | | | | | | | |
| Air Filtration | .5μ | 1μ | 5μ | 15μ | 30μ | 1μ | 10μ | 50μ | 75μ | 3μ | 3μ |
| (ε > 98% @ 50 ft/min) | | | | | | | | | | | |
| Water Filtration | 5μ | 10μ | 15μ | 20μ | 90μ | 10μ | 40μ | 100μ | 150μ | 15μ | |
| (ε > 95% @ 4 ft/min) | | | | | | | | | | | |
| Relative Raw Material | $$$$ | $ | $ | $ | $ | $$ | $$ | $$ | $$ | $$$ | $$$$ |
| Price | | | | | | | | | | | |

The heating disc 100 may be made of a broad combination of the ingredients resulting in a sufficiently rigid and strong molded material that can hold its shape, yet is easily hand moldable to the closed eyelid surface for optimal therapeutic effect. The material porosity allows a heating material to reside in the pathways with access to air at between 10-90 ft/min @1.2″ H₂O ΔP, where the material is between 0.125″ (3.175 mm) and 0.250″ (6.35 mm) thick with enough porosity space to adhere sufficient heating material to the support surface and internal sites. Further, the various ingredients of the disc can be substituted for different materials by shape and size to control the heating rate, total thermal energy converted and delivered, and longevity of the heat conversion.

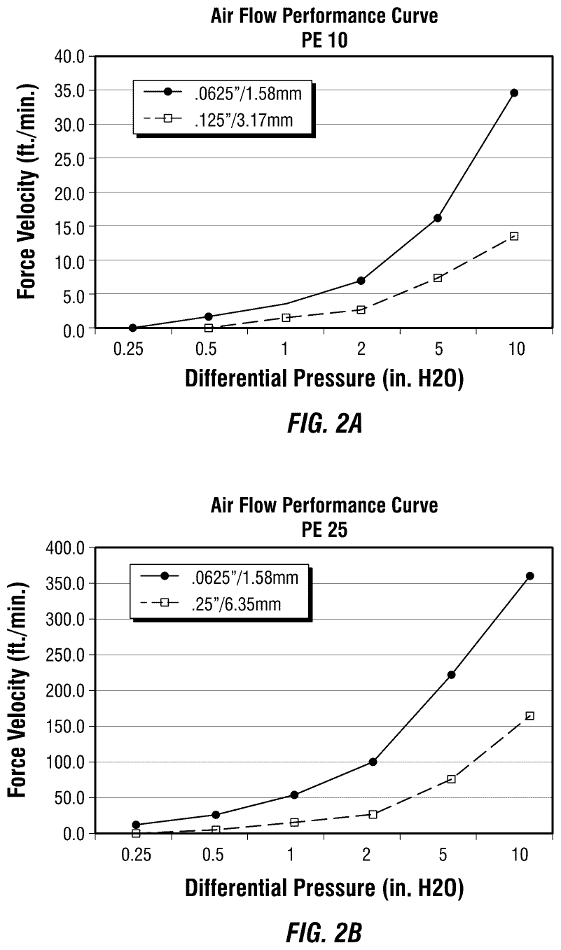

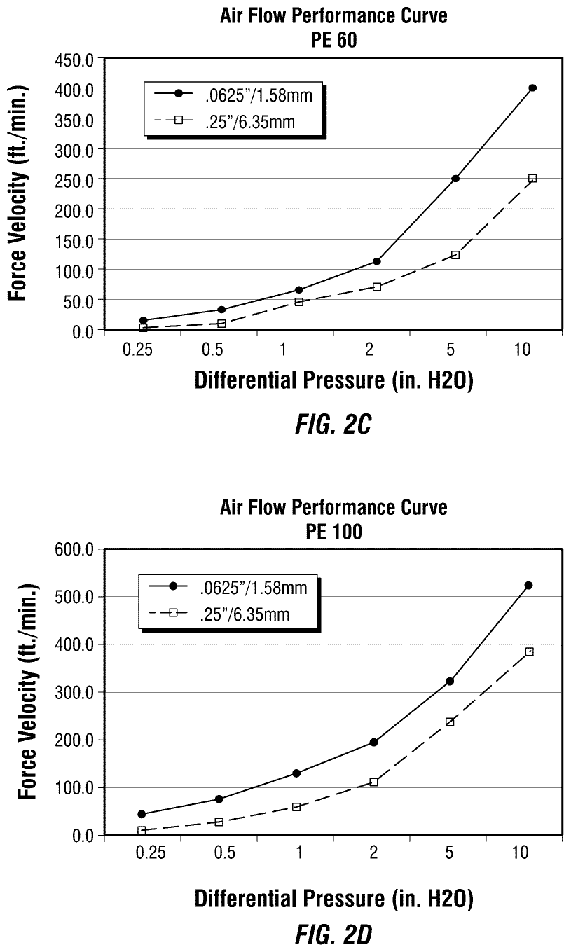

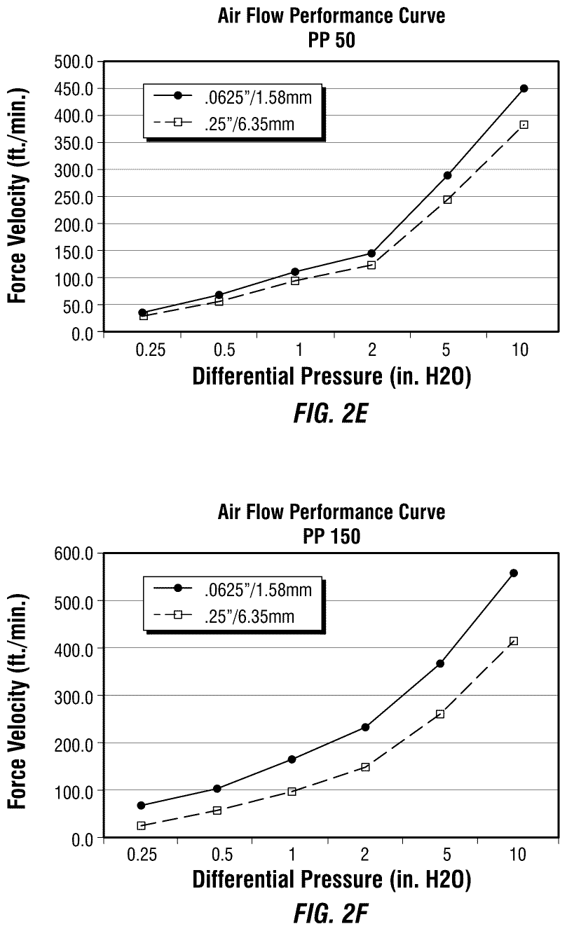

FIGS. 2A-F are graphs of the force velocity and differential pressure for various porous polyolefin compositions. Porous polyolefin compositions are used for the malleable heater disc support material described herein. FIG. 2A shows the air flow performance curve of force velocity (ft/min) and differential pressure (inches H₂O) with a thickness of 0.0625″ and 0.125″ for the PE10 material shown in Table 1. FIG. 2B shows the air flow performance curve of force velocity and differential pressure with a thickness of 0.0625″ and 0.25″ for the PE25 material shown in Table 1. FIG. 2C shows the air flow performance curve of force velocity and differential pressure with a thickness of 0.0625″ and 0.25″ for the PE60 material shown in Table 1. FIG. 2D shows the air flow performance curve of force velocity and differential pressure with a thickness of 0.0625″ and 0.25″ for the PE100 material shown in Table 1. FIG. 2E shows the air flow performance curve of force velocity and differential pressure with a thickness of 0.0625″ and 0.25″ for the PP50 material shown in Table 1. FIG. 2F shows the air flow performance curve of force velocity and differential pressure with a thickness of 0.0625″ and 0.25″ for the PP150 material shown in Table 1.

The construction details of the heating disc 100 shown in FIG. TA and FIG. 1B are that the heating disc material be made of iron converting to ferrous oxide, with salts and inert materials resulting in the following reaction 4Fe(s)+3O₂(g)→2Fe₂O₃(s). The salts and inert materials act as reaction accelerants or retardants as needed to slow or speed the reaction and as dispersants to help create a uniform mixture within the polymer matrix. Once the reaction of 4Fe(s)+3O₂(g)→2Fe₂O₃(s) is driven to completion the individual reaction cannot easily be reversed, so each disk becomes a single use disposable product.

Still referring to FIGS. TA & 1B, there is shown the heating disc 100 with a curvature. Although the disc appears to be solid in nature, there is porosity available to allow access of air to the surface and orifices of the heating material. The chosen porosity along with the ingredient choices and density of the disc when manufactured, and available surface area of the heating material, dictates heat conversion rates.

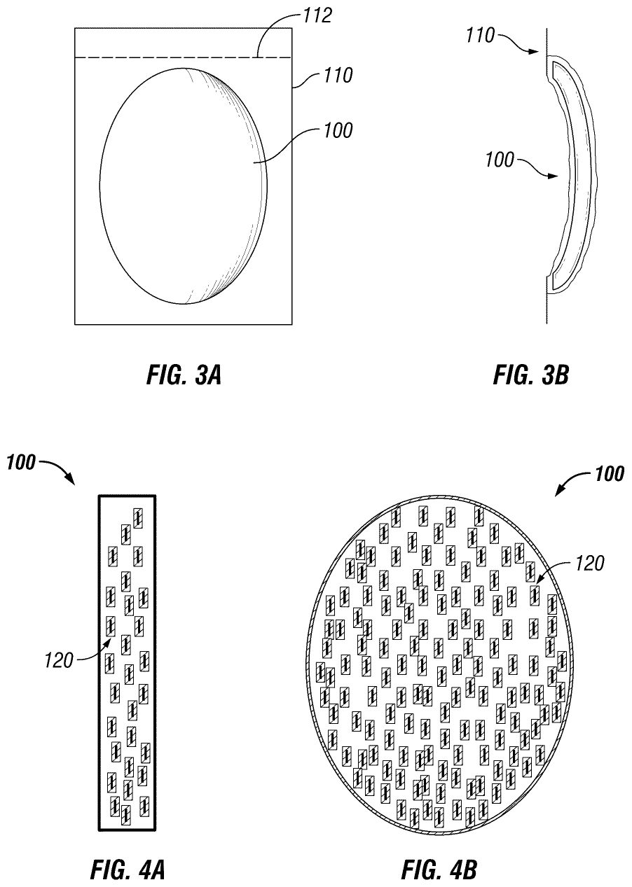

In more detail, still referring to the heating disc 100, the disc shown in FIGS. 3A & 3B includes an overwrap material or barrier layer 110 for shipping and product storage until used by the patient. The barrier layer 110 provides an extended shelf life of at least 3 years and is needed to keep H₂O and air away from the heating disc 100 to prevent premature reaction of the heating material. A pouch tear strip 112 is provided where a user may tear the barrier layer 110 for actual used of the heating disc 100. Water Vapor Transmission Rate (WVTR) measures the transmission of water vapor through a material and is determined using a modulated Infrared Sensor ASTM F1249 Test Procedure. WVTR is measured in either grams/100 in²/24 hours or grams/m²/24 hours (according to the standard ASTM—E398). Oxygen Transmission Rate (OTR) is the measurement of the amount of oxygen gas that passes through a material over a given period of time and is determined using a Coulometric Sensor ASTM D3985, ISO 15105 Test Procedure. OTR is measured in either cm³/m²/24 hours or cm³/100 in²/24 hours. The barrier layer preserves the desired properties of the disc for at least three years. Lower OTR and WVTR rates are preferred. An example of materials with good storage properties are WVTR rates of 0.3 g/100 in²/24 hours @ 37.8° F.=90% RH, for PP and 0.35 g/100 in²/24 hours @ 37.8° F.=90% RH, for high-density PE (HDPE). While very good for WVTR, these have less attractive OTR rates at 150 cm³/100 in²/24 hours @ 25° C., for PP and 110 cm³/100 in²/24 hours @ 25° C., for HDPE. Typical long-term barrier layers 110 also include a layer of aluminum foil. This is a good example of why multi-layer commercial films are used for the product overwraps which include preferred transmission rates for both WVTR and OTR characteristics. When combined, these characteristics achieve longer shelf lives for three-year protection of a product sensitive to water and oxygen.

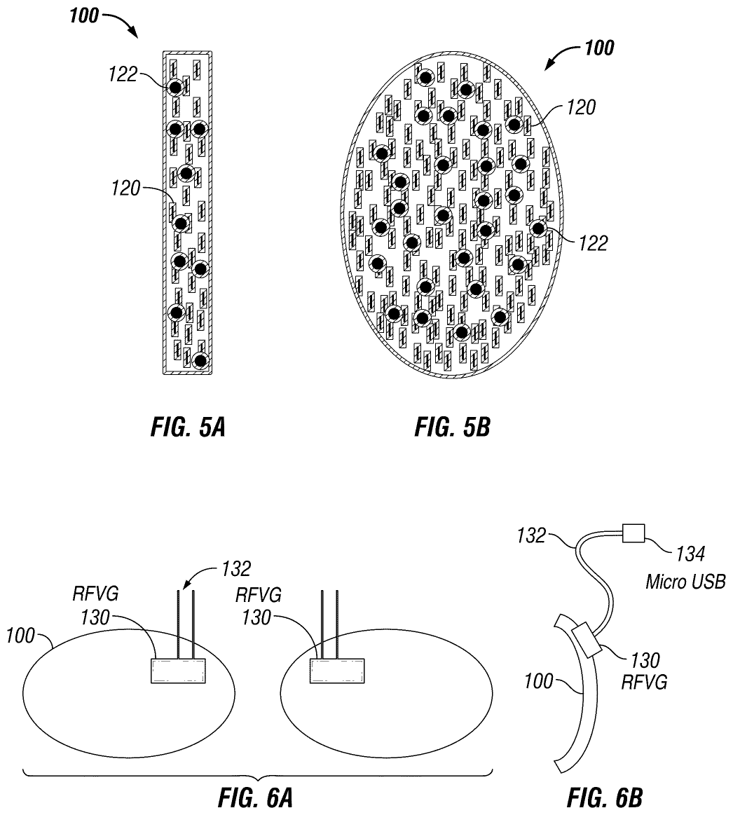

In further detail, referring to the heating disc 100 of FIGS. 4A-4B and FIGS. 5A-5B, the construction of the disc, meaning its shape, materials of construction, ingredient ratios of construction material, surface area, density and porosity make this disc a unique moldable heater product. Furthermore, the heating disk polymer support material, during manufacture, can be infused with nanoparticles of silica or metals to store and then release the heat generated by the FeOₓ heating material. This combination of components in the polymer matrix can be titrated and calibrated to transfer heat rate across the supporting materials and mask at a desired rate. Aluminum or other metal nanoparticles 120 are good heat conductors where the polymer is not. Silica or ceramic nanoparticles 122 act as a heat sink to store and slowly release the heat energy. Adding nanoparticles 120, 122 to the polymer, particularly the metals 120, also enhances desirable vibration transfer characteristics. The nanoparticles 120, 122 participate in structural integrity, also enabling a degree of structural pliability that allows for customized molding of the product to the individual patient's closed eyelid surface topography, as well as greater longevity of use for the product. This also translates into better product survival during transportation and during customer use. Polymer choices with good flexibility, shape memory, permeability and good heat transfer for desirable product characteristics can be determined from Table 1.

According to at least one embodiment, there is provided a heating disc 100 with harmonic resonance frequency stimulation vibration which is designed for use or reuse in a treating physician's office. Referring to FIGS. 6A-6B, the heating disc 100 includes resonance frequency stimulation vibration generators (RFSVG) 130 to transfer shear forces or vibrational energy to the surface of the eyes. Those RFSVGs 130 could be a single unit or one for each eye. The heating disc 100 is designed to hold the RFSVG 130 and fit comfortable over the patient's head and apply gentle pressure sufficient to transfer resonance frequency stimulation energy to the closed eyelids, either to the inner surface of the eyelid or on the entire external eyelid skin surface. To transfer the energy (heat and vibration) directly to the eyelid surface, a sanitary and disposable single use coupling device contacts the patient's skin. Energy and control is provided to the heating disc 100 via lead wires 132 and a micro USB 134 or other suitable connector from a mobile control device.

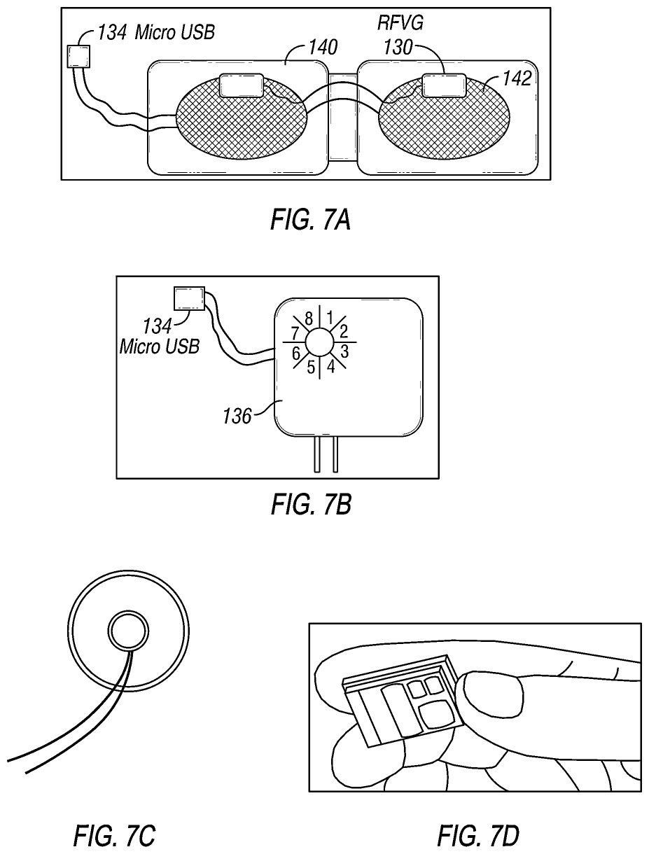

According to at least one embodiment, there is provided a heating mask 140, as shown in FIGS. 7A and 7B, with harmonic resonance frequency stimulation vibration which is designed for use or reuse in a treating physician's office. For purposes of illustration, a resusable heating mask 140 will be described in the following embodiments. Referring to FIG. 7A, the heating mask 140 contains a built-in heater element 142 or individual elements for each eye. The heater element 142 can include the heating disc 100. The system further includes RFSVGs 130 to transfer shear forces or vibrational energy to the surface of the eyelids. Those RFSVGs 130 could be a single unit or again one for each eye. The heating mask 140 is designed to hold these components and fit comfortably over the patient's head and apply gentle pressure sufficient to transfer resonance frequency stimulation energy to the closed eyelids, either to the inner surface of the eyelid or over the entire eyelid surface. To transfer the energy (heat and vibration) directly to the external eyelid skin surface, a sanitary and disposable single use coupling device contacts the patient's skin. Energy and control is provided to the heating mask 140 via a micro USB 134 or other suitable connector from a mobile control device. As shown in FIG. 7B, the heating mask 140 can include a vibration modulation controller 136 connected via USB 134.

The details of the heating mask 140 in FIG. 7A show a built-in resistance heater element 142 or metal wire woven into the mask designed with a heating element length to convert enough electrical input to the appropriate eyelid at optimum temperatures as detailed above. The resistance heater wire or metal wire can be woven into the fabric or structure of the heating mask 140 and has dual purposes: one is to generate heat when electrically energized and the other is to serve as a good vibration conductor. In some embodiments, the heater element 142 can include the heating disc 100. The resistant heater element 142 can be a single, moldable heater mesh having a shape similar to the heating mask 140.

According to at least one embodiment, power supply to the resistance heater can be accomplished for example through the USB connection 134 to a micro drive or mobile control device. Alternate power sources include disposable and rechargeable batteries. These batteries could be placed into the reusable mask if desired to eliminate cords extending from the reusable mask. A micro drive control board controlling the heater and resonator functions could be powered from a single supply voltage of 8-48 VDC, offering up to 100 W of peak power without any additional heat-sink. FIG. 7D is an example control board sold by Ingenia Motion Control. These boards combine the controller, drive, and stand-alone capability into a single unit with an incredibly small footprint. The control board preferably resides in a mobile system controller that is easy to carry around the physician's office. The mobile controller might combine the controller, drive, and stand-alone capability into a single unit with a small footprint. This is but one example of “off the shelf” components available to combine with the heating mask 140 and complete the frequency stimulation set-up and control of the heating mask 140.

According to at least one embodiment, the miniature RFSVG 130 induces a vibration through the coupling device to the surface of the eyelid. The control of vibration may include amplitude, a width, frequency, and where one or more of these parameters may be varied over the treatment period. The resonant vibration may have a frequency stimulation between about 2 Hz to about 270 Hz, between about 15 Hz to about 40 Hz, or between about 30 Hz to about 60 Hz. The resonant vibration may include a current having a pulse width or duty cycle between about 20% to about 80%. Vibration having the above-mentioned parameters may be used to treat one or more conditions, such as dry eye. Ideally in the physician's office, the controller would run through a range of pre-established frequencies and patterns. This range is to determine an individual patient's best response of resonance frequency stimulation to the applied vibration. This resonance frequency stimulation is the condition best suited to excite and mobilize an individual's flow of the Meibum lipids from the Meibomian glands. In some embodiments, anthropomorphic features and other characteristics of the patient, for example, eyelid laxity helps in the determination of the patient's personalized resonant vibration frequency. Non-limiting examples of anthropomorphic features and other patient characteristics include eyelid laxity, eyelid dimensions, eyelid mass, eyelid thickness, patient's race, patient's age, patient's sex, and any history of eyelid surgery. Non-limiting examples of anthropomorphic features and other patient characteristics also include MGD status, such as the percentage of clogged or plugged Meibomian glands, the degree of truncated Meibomian glands, and the quality of the Meibum lipid (i.e., thickness, turbidity, and clarity).

According to at least one embodiment, the tunable RFSVG 130 for the heating mask 140 may be provided by several different sources including sonic generators, electrodynamic or mechanical (such as cell phone vibrators) vibration generators. In some embodiments, the source is relatively quiet and able to deliver the vibrational energy through the disposable patient contacting coupling device to the underlying tissue. According to the concept of finding resonance to the patient's Meibomian glands and blocked oil glands, the frequency stimulation may be adjustable and tunable. There are several miniature vibration devices, such as Adafruit, shown in FIG. 7C, which have the recommended range for patient individual tuning capabilities. This device has a 0-5 VDC range running at 11,000 rpm at the top end at 100 mA. The range may be reduced to 0, by reducing the voltage to the device, for example: 3 VDC®60 mA is linear and at 60% or 6,600 rpm. The power requirement is very low and may be operated by remote supplied energy or in-eye-mask supplied battery power.

According to at least one embodiment, direct heating of the eyelids and adjacent areas may be achieved by weaving a resistance NiChrome heater wire as the heater element 142 into the heating mask 140 as shown in FIG. 7A. The energy to heat the heating mask 140 to the desirable temperature may be provided by a USB connector 134. The resistance across the NiChrome heater wire dictates the heat converted by the amount of energy provided. The heating mask 140 has a compartment, or slip on top of the heater area, for the imbedded RFSVG 130. The heat control from the heater wire 142 can also be controlled by the same type of device controlling the RFSVG 130.

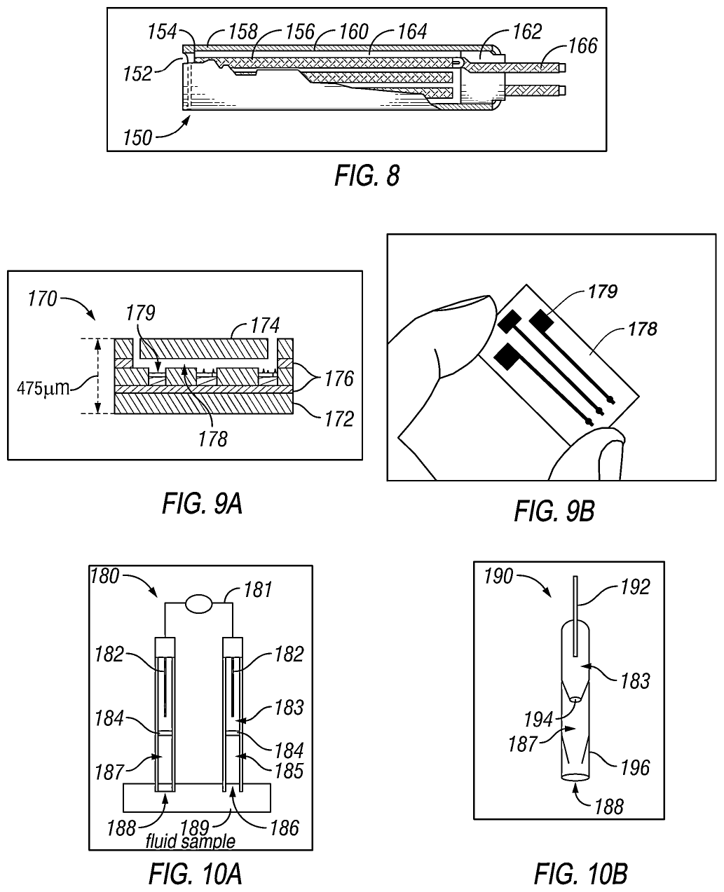

Referring to FIG. 8, the patient's Meibomian gland can also be warmed effectively and comfortably by use of a far infrared front end spot heater 150. Far infrared radiation may be directed to a precise location and the target area warmed to a precise temperature of about 40-46° C. Radiation is the most prevalent source of heat transfer in our universe and the Stefan-Boltzman law of radiation states that as the temperature of a heat source is increased, the radiant output increases to the fourth power of its temperature. Although not to be bound by any theory, this may suggest that far infrared targeted heating is a logical approach to Meibomian gland warming.

According to at least one embodiment, the far infrared front end spot heater 150 is constructed to radiate heat from the far infrared end seal 152 made of heat transmitting material (thin metal face or substitute). Heat is transferred to the far infrared end seal 152 by a conducting plug 154. This plug 154 is in contact with the end seal 152 and is a designed mass of conducting material for storing and releasing the heat converted by a heating element wire 156. The exterior or sides of the spot heater 150 are comprised of heat resistant insulation material 158 allowing a user to comfortably hold the spot heater 150 without risk of uncomfortable temperature exposure. A thermocouple (not shown) might also be employed with this device and integrated into the spot heater 150 properly. The interior of the spot heater 150 includes a conducting packing material 160 all the way to the tip or the plug 154 through a ceramic cap 162. The heating element wire 156 is supported in the spot heater 150 by ceramic element supports 164 that function in a stability capacity providing little movement and adding longevity to the spot heater device 150. The electrical leads 166 are fed through the ceramic cap 162 providing support for the electrical leads 166 and temperature barrier characteristics. The insulated electrical leads 166 are comprised of insulated electrical wire with lead lengths ending in a USB connector 134 for operating the spot heater 150 in the physician's office.

According to at least one embodiment, the heating mask 140 is comprised of soft, comfortable fabric like materials with an adjustable band to help the heating mask 140 reside in the appropriate location on the eyes. A moldable coupling device is a component for the heating mask 140 to provide a sanitary, possibly sterile, skin contacting surface for individual patient use. This single use, disposable coupling device will transfer the generated thermal and vibration energy generated by the heating mask 140 effectively to the eyelid surface. In some embodiments, the coupling device is composed of hydrogel, similar to a hydrogel dressing, possibly contained in a support structure or quilted construction to assure even distribution and intimate contact across the skin contacting regions. The hydrogel composition and water are controlled to best achieve this transfer and add a controlled amount of moisture to the eyelids and lashes, with the added benefit of loosening debris on the eye lashes. According to at least one embodiment, the hydrogel layer makes direct skin contact. In alternate embodiments, the hydrogel could be constrained behind a thin moisture permeable barrier layer. In other embodiments, the coupling device is composed of a hydrogel sheet, and more particularly includes tea tree oil for treatment of, for example, demodex (i.e., mites) infestation of the eyelashes, which is common in blepharitis, Meibomian gland dysfunction, and dry eye disease.

Construction of the coupling device would allow hand molding to an individual's face, periorbita, and features or gentle reforming could be applied from pressure by the eye heating mask 140. The disposable coupling device would be easily replaceable in the heating mask 140 for use by a new patient. The coupling device would be prepared for long term storage using the barrier layer technologies described for the heating disk 100 and could be sterilized to a 10⁻³ or higher sterility assurance level (SAL).

As explained with the heating disk 100 above, this hydrogel layer could incorporate a mixture of particles to facilitate well dispersed heat transfer, heat sinking and bi-directional vibration energy transfer.

Alternatively, the coupling device could be made from thin layers of natural materials and fibers to create a comfortable and breathable surface against the skin. The heating mask 140 could be any number of fiber materials known to be breathable, such as cotton, linen, bamboo, or hemp. Other cloth fabrics from synthetic materials are also breathable and moisture transportable. Non-limiting examples include base layer clothing made from polyester and polypropylene. Filler materials inside the coupling device could be also made of breathable, natural fillers. The filler material may allow the heat to pass to the contact surface but also the vibration energy. Possible natural fillers, in small chunks or fibers, include bamboo fiber, small, dried beans, quinoa, rice, and hemp. Size and size distribution of the filler material can be optimized to determine the best options for transmitting the vibration energy. Also possible are quilted fabric layers using various fillers to provide the loft in the quilt and non-woven felt materials.

According to another embodiment, the coupling device would apply moist heat to the surface. A source for the moist water vapor could be the hydrogel. As heat energy from the heating mask 140 transfers to the coupling device, water in the hydrogel or natural filler turns to vapor and crosses a moisture permeable barrier to the contact surface.

Alternately, reservoirs of water could be constructed into the coupling device to interact with the heat source.

According to at least one embodiment, a microfluidic enabled sensor shown in FIGS. 9A and 9B can be included at the patient's eye lid interface and be responsive in real time to track changes in Meibomian fluid flow. Changes in flow rate are induced by variation in vibration frequency stimulation from the RFSVG; the objective being to determine the best vibration parameters for an individual patient. The sensor provides analysis of very small samples and environments such as the Meibomian gland with the ability to measure very small change in flow. The chemical sensor is an ultra-sensitive, yet simple sensor integrated into a microfluidic device, incorporating polymer-based Meibomian fluid selective liquid-contact and polymer-based solution-selective electrodes. The target component in the Meibomian fluid for the sensor analysis could be specific proteins, lipids, or other biomarkers produced with the flow of the fluid. In-situ sensors enable analysis of very small samples and environments such as the Meibomian gland at work with the ability to realize potentiometric output from very small changes of fluid flow utilizing liquid-contact electrodes, such as electrodes shown in FIGS. 10A and B. It is possible to incorporate the miniature RFSVG and the miniature integrated chemical sensor into the same device.

According to at least one embodiment, one form of the chemical sensor 170, shown in FIGS. 9A and B, has a number of layers formed by polymethylmethacrylate (PMMA) construction. This sensor has a lower PMMA layer 172, a top PMMA layer 174, a sensor liquid entrance, pressure sensitive adhesive (PSA) 176, conducting polymers, thermal pressure laminating, and CO₂ laser-generated 400 μm width fluid flow channels 178. The height of the microfluidic chemical sensor 170 is about 475 μm. A solution reactive material may be needed in the channels for detection on the Meibomian gland fluid selective catalyst layer 179. The deposition of solution reactive materials in the channeling is accomplished by electrode sputtering, if metallic. This process is well known in the art but has yet to focus on Meibomian gland issues as a target for patients until now. In this configuration, the data recorded relates to the presence of fluid.

Referring to FIG. 10A, a miniature solution selective electrode (SSE) 180 with integrated dual chemical sensors with potentiometric output is displayed. The chemical sensor uses a reference electrode 181 consisting of an inner reference half-cell 182, a reference solution 183, a diaphragm 184, bridge solution 185 with the diaphragm 184, a capillary or sleeve 186 at the entrance. The SSE 180 consists of the inner reference half-cell 182, the reference solution 183, the diaphragm 184, an inner filling solution 187, and a solution selective membrane 188. The sleeve 186 and the solution selective membrane 188 are in contact with a fluid sample 189. This miniature chemical sensor, when placed on the surface of the Meibomian gland, detects the duration of flow for patient diagnosis. Electric potential output from the SSE 180 is connected to a data acquisition device (not shown) capable to retrieve data up to 100 Hz speeds, real-time. This may allow real-time data mapping of the patient's Meibomian gland fluid, proteins, biomarkers, or lipids, which all are tracked to define each patient's individual characteristics and medical needs.

According to at least one embodiment, a single element version of the miniature integrated chemical sensor 190 with potentiometric detection, shown in FIG. 10B, is also applicable. It includes a solution sensing wire material 192, the reference solution 183, a separation plug 194, the inner filling solution 187, a SSE body 196, and the selective membrane 188. This single element miniature integrated chemical sensor 190 may be placed on the Meibomian gland and record data at the same rate as the SSE 180.

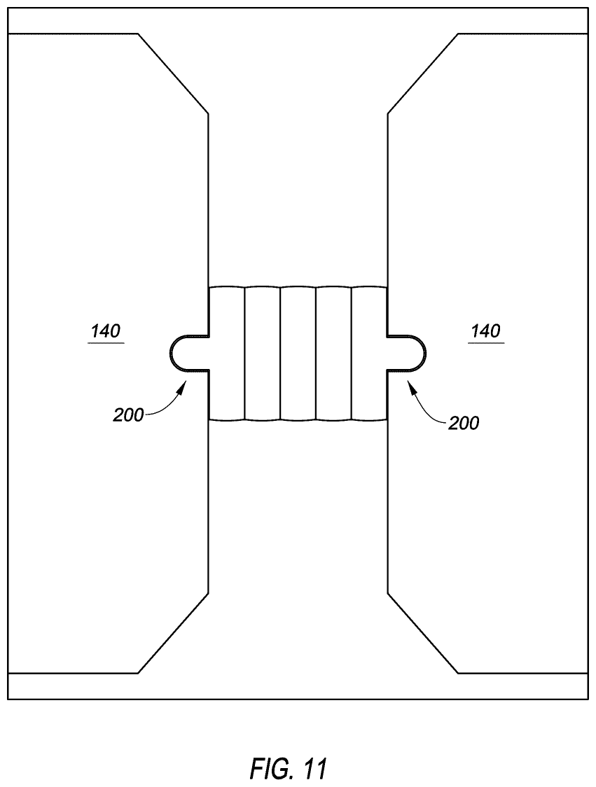

Referring now to the disclosed devices in more detail, in FIG. 11, there is shown the center connecting part of the mask, demonstrating a shape memory bridge 200 component allowing the patient flexibility forming the heating mask 140 to the patient's particular facial and periorbital features and surface topography. This allows better contact surface area by the mask's eye side surface to the eyelid thus achieving better heat transfer to the eyelid surface. According to at least one embodiment, the heating device 100 is manufactured to fit comfortably in the heating mask 140, conforming to the natural shape of the closed eyelid surface, and surrounding periorbital area. One suitable shape is an oval configuration relating to the shape of the ocular surface, other shapes, such as rectangle or square shapes that are larger than the oval shape, are also suitable.

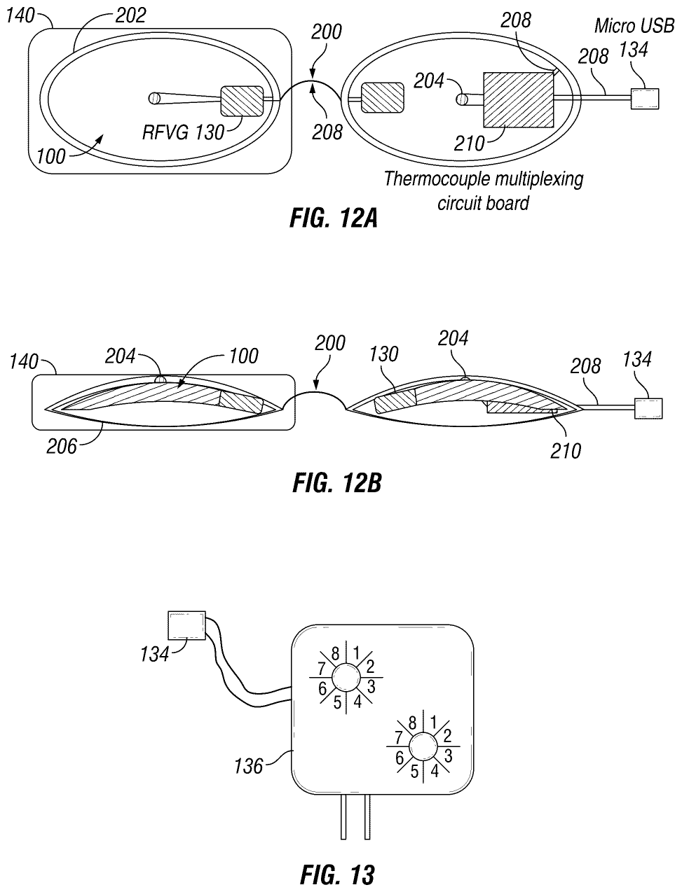

In further detail, referring to the devices disclosed in FIG. 12A and FIG. 12B, there is provided a front and top view of the moldable heating device 100 incorporated in the heating mask 140 with the RFSVG 130 located in one possible location in the moldable heater device 100. The drawing further details the layer of material against the skin 202 allowing better heat and vibration transfer via conductive heat transfer to the skin surface from the heating mask 140. Also shown is the shape memory bridge 200 allowing the patient flexibility forming the heating mask 140 to the patient's particular facial and periorbital features and surface topography. The heating mask 140 configuration shown in FIGS. 12A and 12B incorporates a single thermocouple 204 in each side of the heating mask 140 along with the RFSVG 130. In some embodiments, the mask material located on the outside layer of the heating mask 140 is made of heat reflective material 206 and is purposed to reflect and direct heat from the heating device 100 inward toward the eyelid surface. Reducing heat loss to the outside of the heating mask 140 has advantages such as better temperature control, patient comfort, and overall better temperature distribution over the eyelid surface area. The single thermocouples 204 are imbedded in the inner most layer against the eyelid and record the temperature in that location. The two dissimilar conductors produce a temperature dependent voltage carried via mask wiring 208 to the thermocouple multiplexing circuit board 210 where the signal is conditioned and carried via the USB connector 134 to a remote monitoring device or display for viewing or analysis. In further detail, referring to the devices of FIGS. 12A and 12B, the location of the RFSVG 130 is pre-positioned by the manufacturing process of the heating device 100. The pocket is sized for the RFSVG 130 and associated power sources and connections. The RFSVG 130 can be a reusable electrical device, which snaps into place for the duration of the one-time use or reusable heating device 100 and is then removed before the one-time use or reusable heating device 100 is removed.

FIG. 13 shows a rendition of further capabilities of the RFSVG 130 vibration modulation controller 136 incorporating a temperature controller for the electrical heater element 142, described in FIGS. 7A-7D. The vibration modulation controller 136 can be operated by the user or the physician. Strict control over the electrical heater element 142 allows flexibility over the range of temperatures used in dry eye therapy. The signal is carried out and power is provided via the USB connector 134.

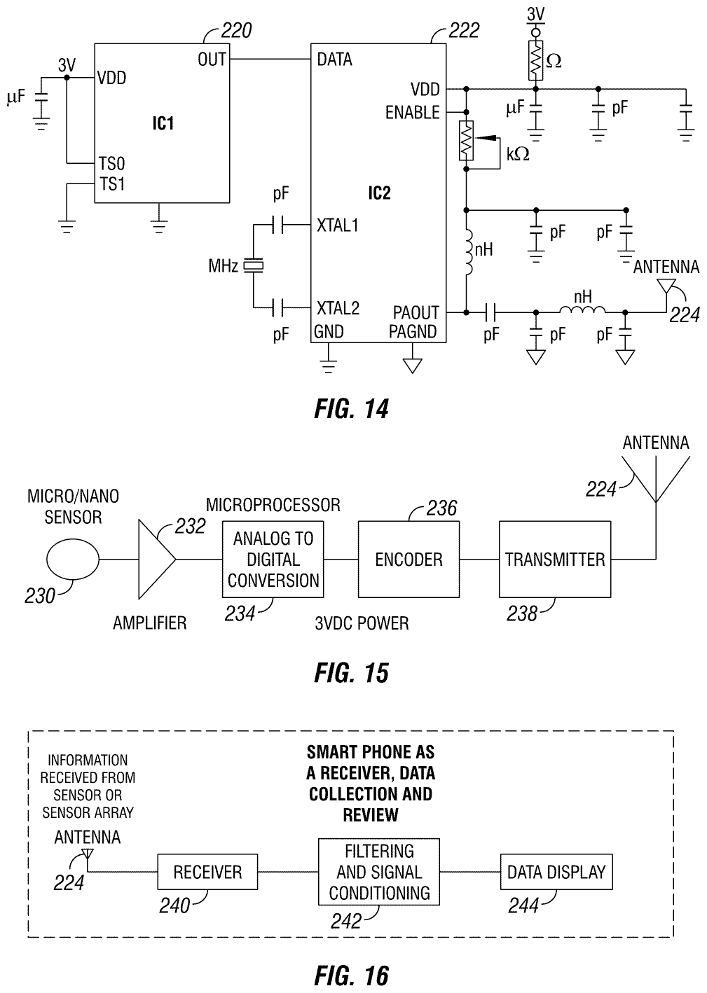

FIG. 14 shows an example electrical schematic showing the circuitry required to localize and transmit sensor information via antenna to a smartphone or smart device receiver, processor, and display. This technology has been well developed and is used to communicate collected data from sensors and sensor arrays via a smartphone or smart device app to a smartphone or smart device receiver, processor, and display. Sensor information collected by IC1 220 is transmitted to IC2 222. IC2 222 processes sensor information and further transmits the processed sensor information wirelessly via antenna 224.

FIG. 15 and FIG. 16 are block diagrams showing the path to transmitting and receiving sensor data to a smartphone or smart device receiver, processor, and display. Block diagrams are included as an assistance feature to better understand or conceptualize the embodiments disclosed herein.

According to at least one embodiment, referring to FIG. 15, analog data generated by the sensors or sensor array 230 is amplified via an amplifier 232 and converted into digital data streams via an analog-to-digital conversion (ADC) microprocessor 234. The digital data streams are encoded via an encoder 236 and then transmitted using a transmitter 238 wirelessly via an antenna 224.

According to at least one embodiment, referring to FIG. 16, wirelessly transmitted data, originated from the sensors or sensor array 230, is received using a receiver 240 via antenna 224 and is then filtered and conditioned for data display 244 using a signal filter and conditioner 242. In some embodiments, a smartphone or smart device is the receiving device. The smartphone or smart device is capable for filtering and conditioning the wirelessly transmitted data and is also capable for displaying the processed data.

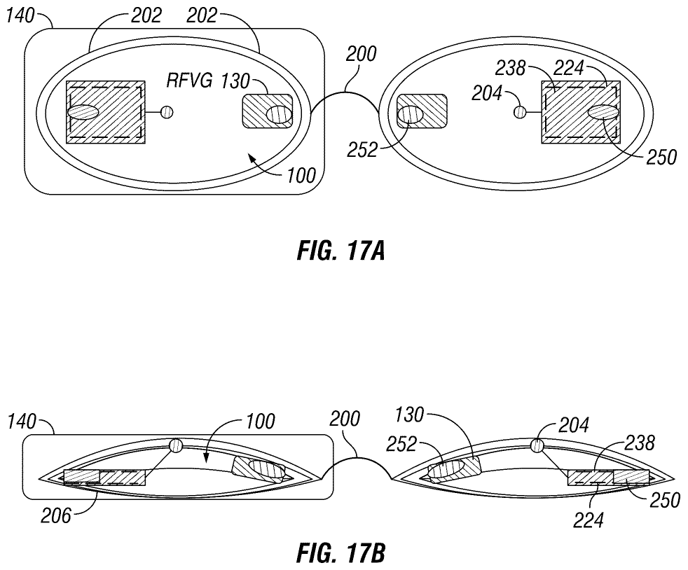

FIGS. 17A and B, still referring to the device in FIGS. 12A and B, show a single temperature sensor 204 location and RFSVG 130 in each ocular surface along with localized transmission power source 250 and RFSVG power source 252 to energize the circuitry and frequency stimulation generator, respectively. In some embodiments, the wireless transmitter 238 is powered locally by a 3 VDC one-time-use or replaceable battery 250 where the thermocouple 204 data is transmitted via an antenna 224 to the patient's smartphone or smart device. The temperature sensor 204 is located in a forward proximity position to the eyelid for reliable and accurate temperature data collection. In some embodiments, the shape memory bridge 200 has no additional electronic connections in the wireless transmittal version. This configuration could also employ a remote power source via USB 134 where the shape memory bridge 200 would carry additional circuitry, corresponding to mask power and communication wiring 208, to transmit the temperature data.

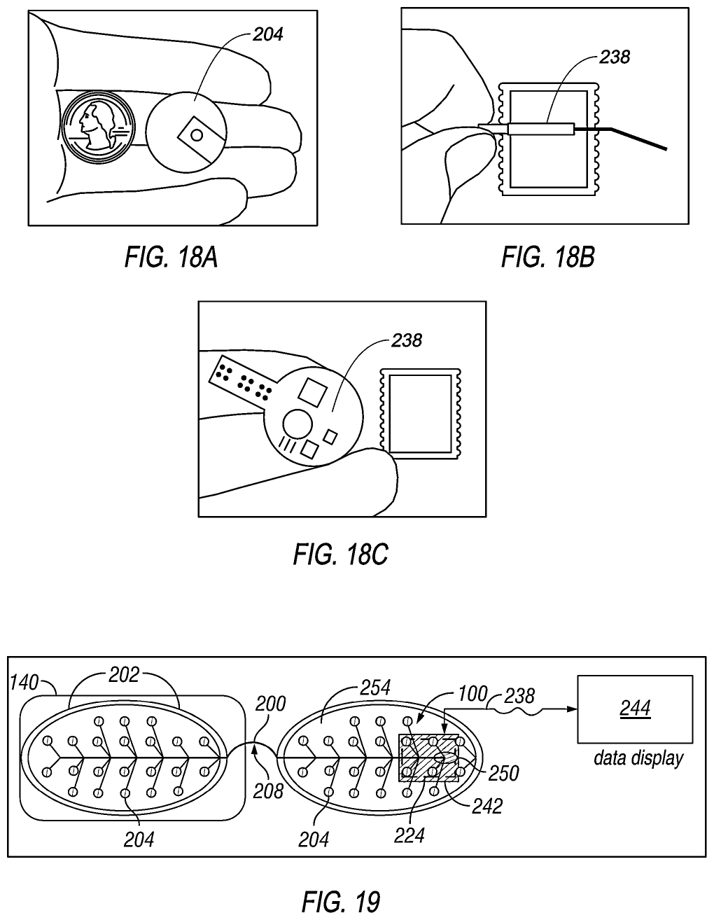

FIGS. 18A-18C show photographs of the microtechnology for collecting temperature data, for conditioning and transmitting the data to a smartphone or smart device. FIG. 18A shows the single thermocouple 204 next to a quarter for size comparison. FIG. 18B shows a side view of the data transmitter 238. FIG. 18C shows a front view of the data transmitter 238. Postage stamps are displayed for size comparison in FIGS. 18B-18C. The micro-components are easily integrated into the heating mask 140 for enhanced characteristics and utility of the proposed device.

FIG. 19, still referring to the device in FIGS. 12A-12B, shows a configuration with multiple thermocouple 204 positions, signal conditioning 242, and wireless transmission 238 of temperature data to a smartphone or smart device, wireless receiver 240, processor, and data display 244. Also provided is a flexible printed circuit board 254, which includes the thermocouples 204. In some embodiments, taking more temperature points in the given area provide a better temperature profile of the amount of energy and energy distribution produced by the heating device 100. The heating device manufacturer may focus on a more precise heater product and tailor the heater device 100 to the individual patient. In other embodiments, still referring to FIG. 19, a micro-frequency stimulation generator, such as the RFSVG 130, may be incorporated, which may be powered by a remote or localized power source, a replaceable battery, or USB connection 134.

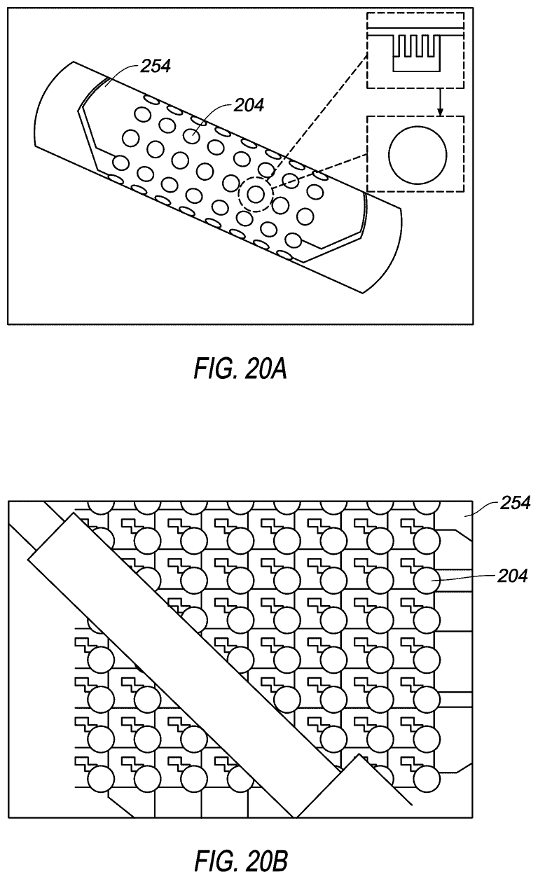

FIGS. 20A and 20B show a flexible printed circuit board 254 with closely aligned temperature sensors 204. This embodiment demonstrates how flexible the heating device 100 may be and how it may be used in the heating mask 140. In some embodiments, graphite-polydimethylsiloxane composite is dispensed on flexible polyimide films. A sensor array 204 may be, for example, an array with 64 sensing cells in a 4×4 cm² area. Interdigitated copper electrodes are patterned on the flexible polyimide substrate to determine the resistivity change of the composites subjected to ambient temperature variations. The flexible circuit operates with no signs of degradation in temperatures from about 30° C. to about 110° C.

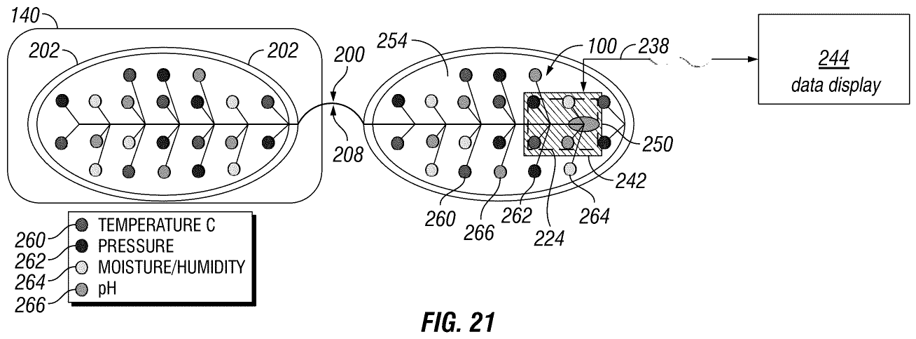

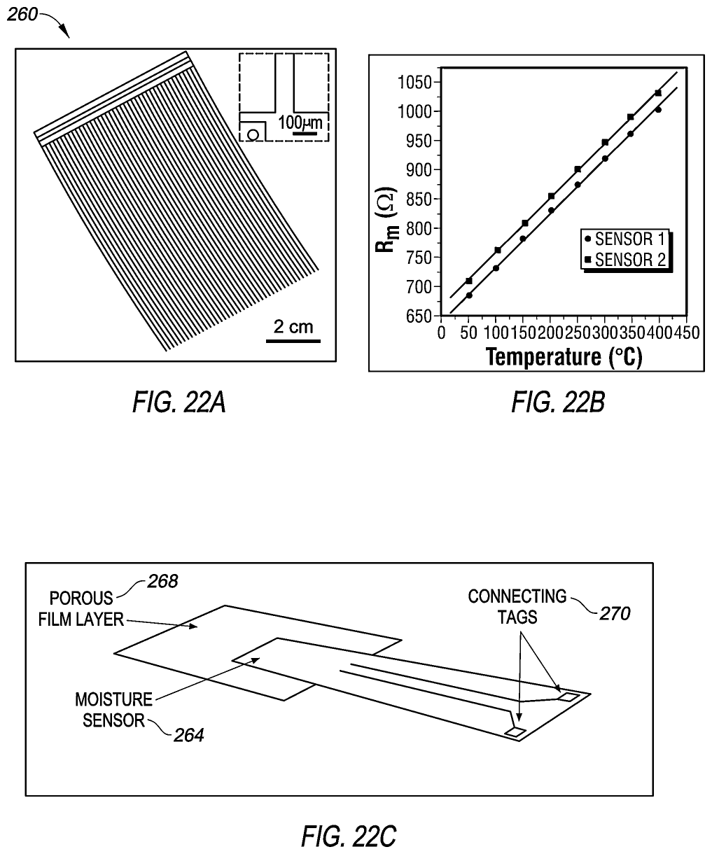

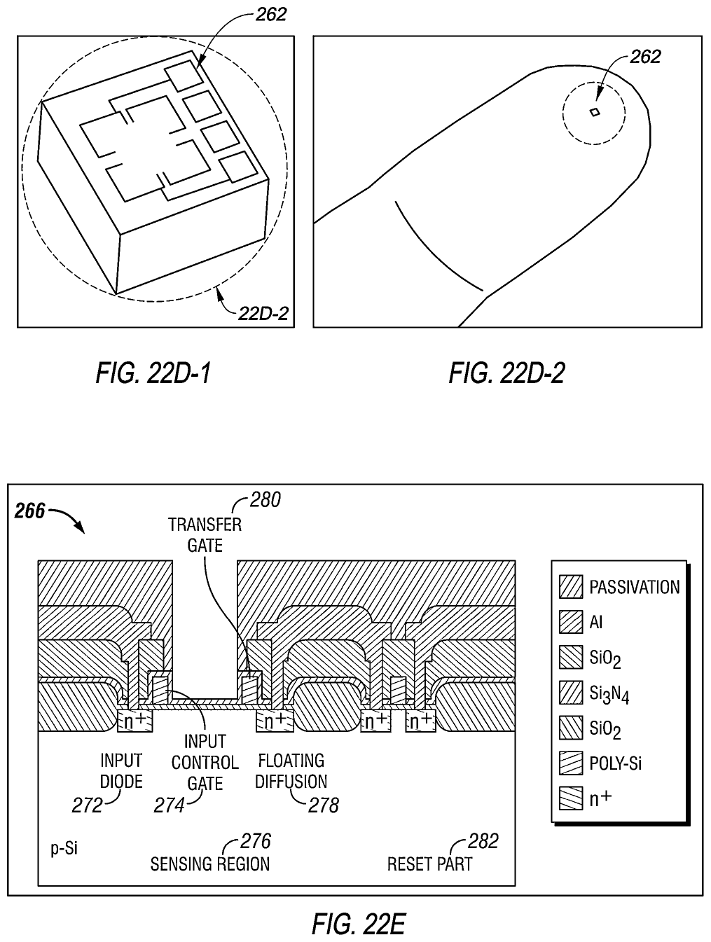

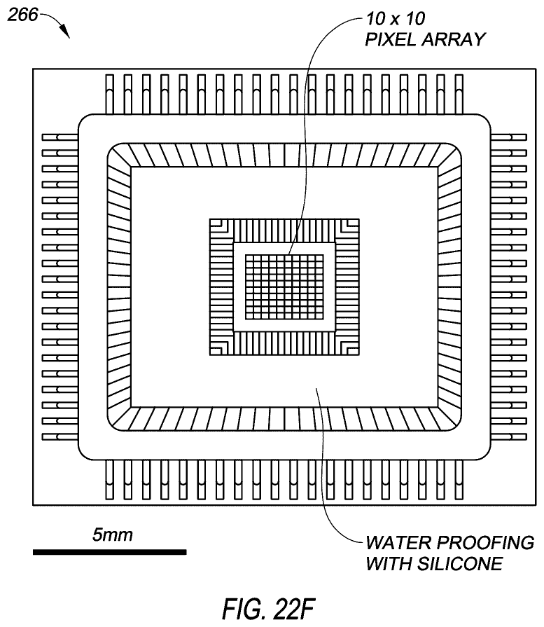

FIG. 21 still referring to the device in FIGS. 12A-12B, shows, in further detail of multiple sensor application, an embodiment where multiple types of sensors are imbedded on a single array 230 of a flexible printed circuit board 254. FIGS. 22A-F show embodiments of various types of sensors 230 that are capable of measuring, conditioning, and transmitting data for temperature 260, pressure 262, moisture/humidity 264, pH 266, and vibration frequency stimulation parameters. Application of these multiple types of sensors has been demonstrated for wound management. In some embodiments, data generated and collected from all of these sensors is conditioned and transmitted using wireless technology to a smartphone or smart device via an app. Sensors 230 are miniaturized for use in the ocular region via the heating mask 140.

Temperature sensors 260 used in embodiments of this disclosure is depicted in FIG. 22A. FIG. 22B shows a performance graph of the miniaturized temperature sensing device 260. As shown in FIG. 22B, electrical resistance (in ohms) of the temperature sensor 260 corresponds with the temperature. In some embodiments, temperature sensors 260 include a printed miniature format showing connection leads for data acquisition beyond the printed circuit board 254.

Referring now to FIG. 22C, an embodiment of a moisture/humidity sensing device 264 used in the mask 140 is shown. Moisture is an important parameter considering dry eye syndrome, sensing a patient's moisture level would be helpful for the physician to address the need whether to add additional moisture to the treatment. Moisture plays an important role in determining improvement for dry eye syndrome. In some embodiments, an additional layer of an aqueous solution provided and measured by micro-sensors 264 would benefit the treatment as adequate fluid balance is achieved. A continuous moisture level measurement provides informed decisions about treatment. In some embodiments, moisture is measured by a pair of replaceable, one-time use silver chloride electrodes. After a period of about 10 to about 30 seconds, the sensor 264 measures the moisture level by recording the impedance value shown in the onboard electronics. The porous film layer 268 surrounding the sensor is bi-functional where the porous film layer 268 allows moisture to reach the sensor 264 and provides mechanical support for the sensor 264 in-situ. Data generated is then transmitted via connecting tags 270 to a smartphone or smart device for processing and display. In some embodiments, a replaceable sensor is used when recommended as part of the sensor array 230.

Referring now to FIG. 22D-1, a pressure sensor 262 is shown including a miniaturized diaphragm-based silicon pressure sensor. A Wheatstone bridge unit is incorporated using silicon-on-insulator technology. Pressure applied to the surface of the closed eyelids is a parameter for determining patient comfort and mask tightness when treating dry eye syndrome. There are several miniaturized pressure devices 262 being manufactured in industry and size is of concern when putting the multiple sensor array 230 into use. FIG. 22D-2 shows a photograph of a miniature pressure sensing device 262 that is powered by a small local source and provides data to a smartphone or smart device via wireless technology. On the right-hand side of FIG. 22D, the miniature pressure sensing device 262 is placed on a fingertip for size comparison.

FIG. 22E shows a depiction of a miniaturized pH sensing device 266. FIG. 22F shows a photograph of the miniaturized pH sensing device 266, where the areas other than the 10×10-pixel array are waterproofed with silicone. A wide range of pH measurement approaches are available, such as pH-sensitive polymers, ion sensitive field-effect transistors, near infrared spectroscopy. In some embodiments, a two-dimensional pH image sensor is manufactured by employing a CMOS fabrication process. Referring to FIG. 22E, the pH sensing device 266 includes an input diode 272, input control gate 274, ion sensing region 276, floating diffusion region 278, transfer gate 280, reset switch 282 and a source follower circuit. Employing CMOS technology, as shown in FIG. 22E, layers of poly-Si, SiO₂, Si₃N₄, Al, and a passivation layer can be deposited and patterned on a base substrate. In some embodiments, an RFID-based wireless optical chemical pH sensor 266 with contactless power and data interface may be employed. The optical chemical pH sensor 266 is based on the differential absorbance of light in bromocresol green (BCG) in which BCG is pH sensitive.

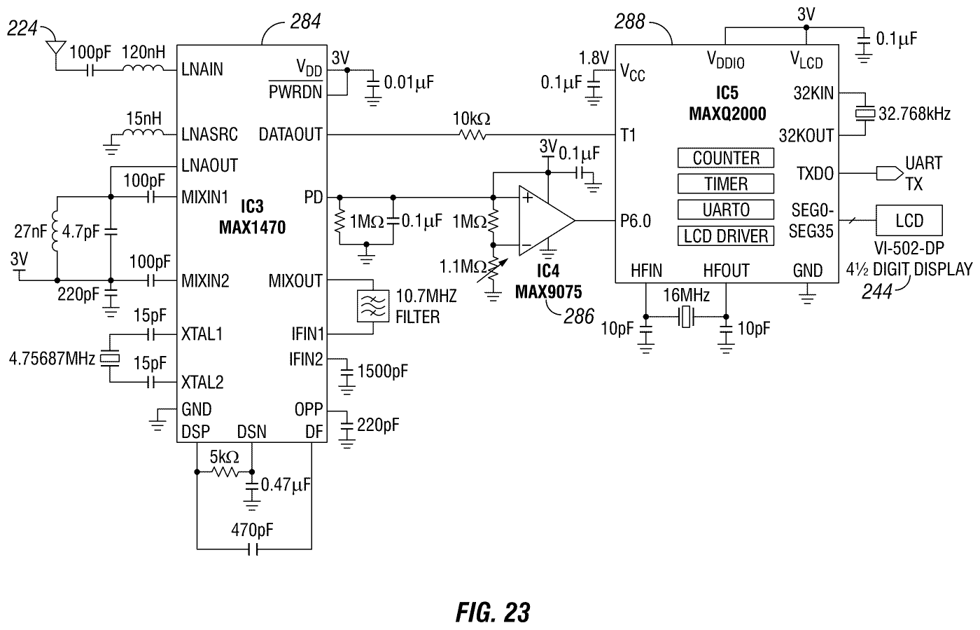

Referring to FIG. 23, an exemplary commercial wire schematic is shown for a smartphone or smart device to receive process and display data from a wireless sensor array. Digitally converted and wirelessly transmitted sensor data is received by IC3 284 via antenna 224. The data is further processed by IC3 284, IC4 286, and IC5 288. IC5 288 transmits the processed sensor data to the display 244 for visual display. Those skilled in the art would recognize that various means exist for a smartphone or smart device to receive, process and display information from a wireless sensor array.

According to at least one embodiment, vibration and temperature are controlled by pulse width modulation (PWM). Switching-voltage regulators employ PWM control for the switching elements. The PWM signal is either generated from a control voltage (derived from subtracting the output voltage from a reference voltage) combined with a saw tooth waveform running at the clock frequency for the voltage-mode regulator, or by adding a second loop feeding back an inductor current for current-mode control. Devices employ techniques such as voltage feed-forward for voltage-control designs and slope compensation for current-mode units.

In some embodiments, both types of topologies are employed in the system. In other embodiments, component parts are linked together in the system. Voltage-mode control switching regulators are used in some embodiments when wide-input line or output-load variations are desired, under light loads (when a current-mode control-ramp slope would be too shallow for stable PWM operation), in noisy applications (when noise from the power stage would find its way into the current-mode control feedback loop), and when multiple-output voltages are needed with good cross regulation.

In some embodiments, current-mode control devices are used for applications where the supply output is high current or very-high voltage; the fastest dynamic response is sought at a particular frequency, input-voltage variations are constrained, and in applications where cost and number of components must be minimized as in the innovations stated here within.

According to at least one embodiment, the reusable mask 140 and even the entire system is suitable for mobile control, in which the device is easily handheld and carried for patient use. Control may also be driven by a smartphone or smart device using operating systems such as iOS, Android or Windows mobile, or other similar interfaces. Mobile medical interfaces are used in products such as a Zebra MC40 Mobile Computer. Similar platforms, or other Wi-Fi, cell phone or Bluetooth connected interfaces can be used to control the patient's first in-office use of the system. In some embodiments, a range of frequencies are tested and output data from the sensors is stored. The data storage and its associated algorithm may determine the best treatment mode for following office visits or transfer an optimal program to an at-home unit. These mobile interfaces further create efficiencies for the office staff by automatically storing patient records to the electronic medical records (EMR) of the first and subsequent uses. These records include patient name, time and date of use, frequencies explored, and sensor output during that time. The at-home unit would also serve as a record of patient compliance to prescribed therapy.

According to at least one embodiment, the harmonic resonance heating mask 140 is preferentially supplied as a kit. Kits include one or more devices, and varying numbers of replacement heaters depending on kit size. Kits may include both elements of the one-time use components and reusable components. In some embodiments, for example, a kit might include the one-time use heating element 100, the reusable miniature harmonic resonance frequency stimulation generator 130 pairs that fit into the eye patch component and plug into a USB 134 port and the one-time use coupling device. Kits may be provided to a patient during an office visit as the equipment used to define the correct resonance frequency stimulation would be available in the practitioner's office. Commercial kits may also be provided with very specific frequencies and then purchased directly by an informed customer.

According to at least one embodiment, as mentioned in describing the mobile controller, after a patient's first use of the system in the physician's office, the patient may be prescribed to continue therapy on a more frequent basis at home. As an alternate embodiment, this system could be simplified for the home user. This system would have a reusable mask 140 with single use disposable, or reusable built-in heating elements 100 and resonance frequency stimulation generators 130, accommodate an optional disposable coupling device and come with appropriate power supply and control, including a mobile and wirelessly connected controller. The at-home monitoring system would not require a full range of vibration frequencies as the optimal frequency stimulation and pattern was determined in the original office use and that pattern is programmed into the individual user's system. Similarly, the full sensing capability is not needed for home use. A cell phone, Wi-Fi or Bluetooth connected controller may also create a record of use for the patient's EMR. In some embodiments, patterns of noncompliance or misuse may create an alert to go directly to the patient and/or back to the treating physician.

A further alternate embodiment may include a system that employs single use heating discs 100. This could be used for either the office based or home use products. The disposable heating disc 100, being hand moldable to conform to an individual's anatomy, would fit into the pocket in the heating mask 140. This heating disc 100 element could also be built into and supplied as part of the coupling device that contacts the skin and comprises a combined single disposable item. As shown in FIGS. 6A and B, in some embodiments, a moldable heating disc 100 with a low cost RFSVG 130 may be located in the disposable heating disc 100 and connected via the USB 134 port for control and energy. A reusable RFSVG 130 (mini-vibrator) can be placed in the manufactured pocket location located in the moldable heating disc 100. This approach may be used for both the moldable gel and polymer eye piece.

According to at least one embodiment, there is provided a method of treating dry eye disease or MGD. These methods include the initial physician's office-based use where optimal treatment parameters are determined and then stored for later use either in subsequent office visits or home use.

The advantages of the devices disclosed include, without limitation, that it is portable, easy to transport, reliably functions as intended, and is simple and convenient to activate and use. Another advantage is that it is easy to integrate these devices into a reusable face mask or eye patch because they are relatively small and lightweight, showing the parallel utility of the device components stated herein.

A further alternate embodiment may include an integrated real-time imaging device to detect optimal tuning of the RFSVG 130 to the particular patient eyelid and Meibomian glands. In some embodiments, for example, optoacoustic imaging or photoacoustic imaging is insensitive to photon scattering within biological tissue and, unlike conventional optical imaging methods, makes high-resolution optical visualization deep within tissue possible. A key empowering feature is the development of video-rate multispectral imaging in two and three dimensions, which offers fast spectral differentiation of distinct photo-absorbing moieties. In some embodiments, the imaging device provides a real-time-image-based assessment of the optimal settings for the miniature RFSVG 130 at which there is maximal movement of the eyelids, Meibomian glands, and lipid fluid within the Meibomian glands.

According to at least one embodiment, there is provided means for providing a physician and a patient with a metric related to the state of the dry eye disease being treated. This metric will correlate to the severity of disease and may be measured and provided both before and after treatment. Increased sensitivity to light is a well-known proxy for severity of dry eye disease. According to at least one embodiment, there is provided a light sensor configured to measure light sensitivity of the eye being treated and to provide a subjective light sensitivity score as a diagnostic indicator.

According to at least one embodiment, there is provided a method in which prior to initiating a treatment, the patient looks at a target in the mask 140 or at a distance. A light-emitting diode (LED) with a controllable spectrum is mounted to a head-mounted mask. The LED in the mask will turn on at an adjustable initial setting. The patient adjusts the intensity to the maximum comfortable level, with a physical rheostat or other controller. Right and left eyes may be tested sequentially, or both eyes may be tested simultaneously. The light intensity setting is recorded electronically. At the end of the treatment, the patient is exposed to light and the light sensitivity measurement is performed again. Each time the patient uses the device, their pre-treatment and post-treatment light sensitivity is recorded electronically, and comparison made with the previous light sensitivity scores. The comparison provides an indicator of treatment success, as well as dry eye disease stability, improvement, or worsening. According to at least one embodiment, the system can include a feature to automatically increase or decrease the treatment duration and/or intensity based on the light sensitivity measure, and relative change from the previous light sensitivity value.

According to at least one embodiment, the heating mask 140 can be configured to fit a single eye. The single-eyed heating mask 140 can be configured to fit either the patient's right or left eye. In some embodiments, the heating mask 140 can include two single-eyed heating masks, one configured to the right eye and the other configured to the left eye.

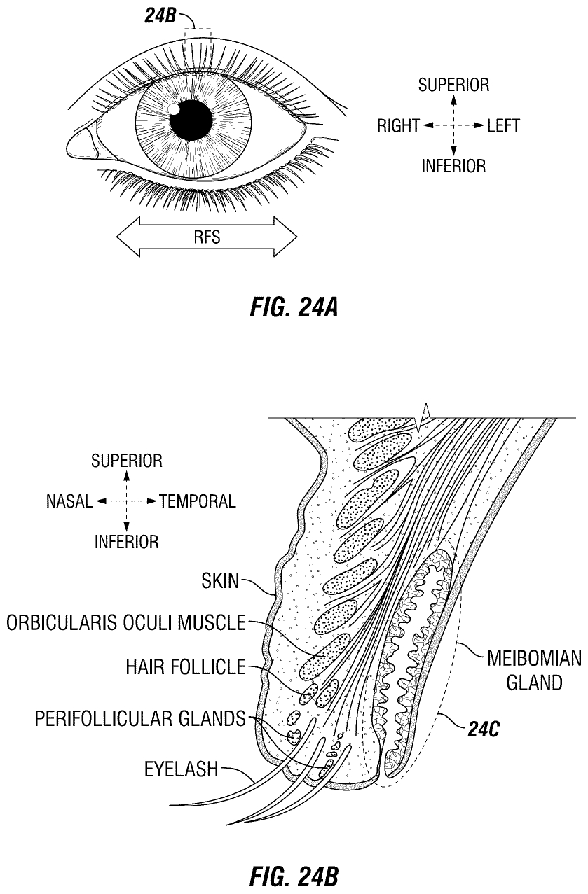

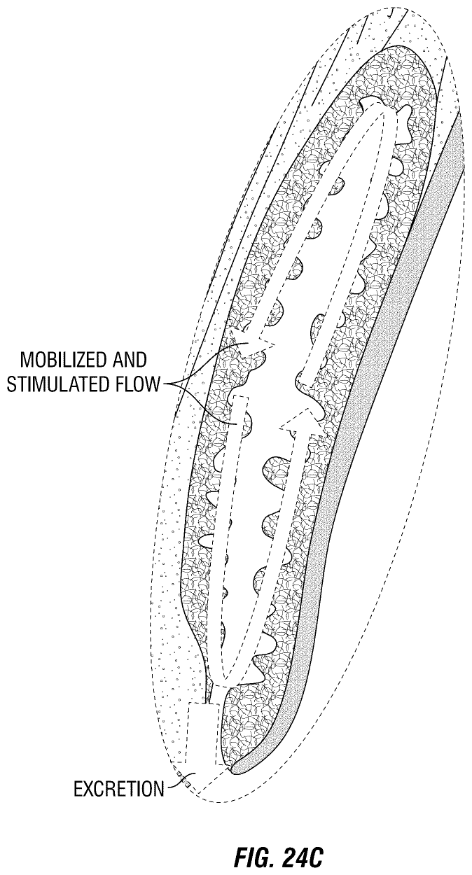

FIG. 24A shows a front view of a human eye. FIG. 24B shows an enlarged cross-sectional view of an upper eyelid of the human eye. FIG. 24C shows a cross-sectional view through a Meibomian gland of an upper eyelid of a human eye where the harmonic resonance frequency stimulation vibration mobilizes and stimulates flow of the Meibum lipids within a Meibomian gland. The mobilized and stimulated flow leads to enhanced excretion of the Meibum lipids.

According to at least one embodiment, the harmonic resonance frequency stimulation is a vector force. The harmonic resonance frequency stimulation exhibits traits substantially like a longitudinal wave. In some embodiments, the harmonic resonance frequency stimulation exhibits traits substantially like a longitudinal standing wave.

According to at least one embodiment, the primary axis of vibration is substantially parallel to the medial-lateral axis, as shown in FIG. 24A. The primary axis is tangential to the depth of the Meibomian gland duct, which is shown as the superior-inferior axis in FIGS. 24A and 24B. As shown in FIG. 24B, the primary axis of vibration is substantially tangential to the opening of the Meibomian gland duct. In some embodiments, the amplitude of the vibration, corresponding to the vector force, is configured to be substantially parallel to the medial-lateral axis, as shown in FIG. 24A. Accordingly, a RFSVG 130 may generate harmonic resonance frequency stimulation vibrating in the medial-lateral direction, which is substantially tangential to the depth of the Meibomian gland duct.

According to at least one embodiment, the primary axis of vibration can be substantially parallel to the superior-inferior axis, as shown in FIGS. 24A and 24B. The amplitude of the vibration, corresponding to the vector force, is configured to be substantially parallel to the superior-inferior axis, as shown in FIGS. 24A and 24B. Accordingly, a RFSVG 130 may generate harmonic resonance frequency stimulation vibrating in the superior-inferior direction, which is substantially parallel to the depth of the Meibomian gland duct.

According to at least one embodiment, the harmonic resonance frequency stimulation can be a superposition of two or more longitudinal vibrational waves. In some embodiments, the harmonic resonance frequency stimulation is a superposition of two longitudinal vibrational waves, where the primary axis of the first vibration can be substantially parallel to the superior-inferior axis while the primary axis of the second vibration can be substantially parallel to the medial-lateral axis, both axes as shown in FIGS. 24A and 24B. The amplitude of the first vibration, corresponding to the first vector force, is configured to be substantially parallel to the superior-inferior axis, as shown in FIGS. 24A and 24B. The amplitude of the second vibration, corresponding to the second vector force, is configured to be substantially parallel to the medial-lateral axis, as shown in FIG. 24A. Accordingly, a RFSVG 130 may generate two simultaneous harmonic resonance frequency vibration stimulations, one vibrating in the superior-inferior direction, which is substantially parallel to the depth of the Meibomian gland duct, and the other vibrating in the medial-lateral direction, which is substantially tangential to the depth of the Meibomian gland duct.

According to at least one embodiment, the direction of the harmonic resonance frequency stimulation vector force can be selected by the device operator based on the degree of MGD (i.e., truncated gland ducts, clogged or plugged gland orifices versus open gland orifices).

According to at least one embodiment, the harmonic resonance frequency stimulation liquifies and mobilizes the Meibum lipids within the Meibomian glands. The mobilization is achieved by inducing shear forces using vibration at the resonance frequency or frequencies of the patient's eyelid and Meibomian gland complex. As shown in FIG. 24C, a flow of the mobilized Meibum lipids is generated as a result of the vibration. After the Meibum lipids are mobilized, natural eye blinking will lead to the excretion of these lipids.

According to at least one embodiment, the reusable mask can include a bladder-type coupling device to provide substantially full contact to the patient's individual eyelid and periorbital three-dimensional anatomy and surface topography. The bladder-type coupling device can be filled with gaseous or fluidic medium, or foam. Hydraulics or pneumatics can be applied to control the coupling device. In some embodiments, a hydraulic medium is used in the bladder-type coupling device, where the hydraulic medium has a viscosity suitable for conforming to the patient's individual eyelid and periorbital three-dimensional anatomy and surface topography. The hydraulic medium is suitable for transmitting harmonic resonance frequency stimulation vibration generated by the RFSVG 130 to the patient's Meibomian glands. The hydraulic medium is suitable for transmitting the vibration in any direction. In some embodiments, the bladder-type coupling device includes channels to provide direction control of the vibration. In some embodiments, temperature and pressure control of the reusable mask can be achieved by hydraulically inserting the hydraulic medium into the bladder-type coupling device.

According to at least one embodiment, the heating mask 140 is operable to change configuration of applying heat and the resonance frequency stimulation vibration. In some embodiments, the heating mask 140 is operated such that heat (for example, provided by the heating disc 100) and the resonance frequency stimulation vibration (for example, provided by the RFSVG 130) are applied to the patient's eyelid area sequentially and alternately, but not simultaneously. The heating mask 140 can be internally or externally programmed to achieve this sequence. In some embodiments, the medical practitioner cannot override the programmed sequence. In other embodiments, explicit instructions, such as an instruction manual, can be given to the medical practitioner to operate the heating mask 140 by applying heat and the resonance frequency stimulation vibration sequentially and alternately.

FIG. 25 shows an embodiment of the heating mask 140 including a neurostimulation unit 510. The heating mask 140 includes the moldable coupling device 520. In some embodiments, the moldable coupling device 520 includes hydrogel to assure even distribution and intimate contact across the skin contacting regions. The heating mask 140 conforms to the three-dimensional topography of the eyelid surface. The heating mask 140 serves as a heat reservoir to deliver heat to the Meibomian glands through the eyelids. In some embodiments, the heating mask 140 includes the RFSVG 130 (not shown) to transfer shear forces or vibrational energy to the surface of the eyelids. Resonant frequency or non-resonant frequency stimulation vibration is delivered over the eyelids in a uniform manner to cause liquefaction and mobilization of the Meibum within the Meibomian glands.

According to at least one embodiment, the neurostimulation unit 510 includes the RFSVG 130 to transfer shear forces or vibrational energy to the nasal bridge area located between the eyes (as opposed to the cartilage area of the nose). Resonant frequency or non-resonant frequency stimulation vibration is delivered, based on determined tuning parameters, across the contour of the nasal bridge area through the nasal bone and to the sensory nerves (such as the anterior ethmoidal nerve in the nasal septum) to induce neuromodulation of the tear production reflex in the lacrimal functional unit (LFU), which encompasses the epithelium of the cornea and conjunctiva, the main and accessory (Wolfring and Krause) lacrimal glands, Meibomian glands, conjunctival goblet cells, and its corresponding innervation, to thereby induce expression of shear thinned and liquified meibum from each Meibomian gland in all four eyelids to improve the lipid layer of the surface of the user's eye tear film. Without being bound by any theory, stimulation in the nasal sensory nerves serves as an alternate afferent pathway for tear production reflex stimulation in addition to stimulation in the sensory nerves on the ocular surface. Stimulation is received in the sensory receptors located at the nasal mucosal epithelium, and travels to the superior salivatory nucleus through the anterior ethmoidal nerves, which is a branch of the ophthalmic division of the trigeminal nerve (i.e., the 5^{th} cranial nerve). The stimulation continues to travel through the pre-ganglionic fibers along the nervus intermedius to the pterygopalatine ganglion, and innervate the lacrimal glands, goblet cells, and the Meibomian glands. Such stimulation in the lacrimal gland results in an increase of the aqueous component of the tear, which is the middle layer component of the tear film. Such stimulation in the goblet cells on the conjunctiva results in the production of mucin, which is the innermost component of the tear film. Such stimulation in the Meibomian gland induces expression of shear thinned and liquified meibum from each Meibomian gland in all four eyelids to improve the lipid layer of the surface of the user's eye tear film.

Certain parameters of the resonant frequency stimulation vibration can be adjusted for optimal neurostimulation. For example, frequency can be adjusted such that the resonant frequency or non-resonant frequency stimulation vibration is optimized for bone conduction around the nasal bridge. Amplitude can be adjusted for optimal comfort and effectiveness. The primary axis of vibration can be substantially parallel to the medial-lateral axis, as shown in FIG. 24A, which is perpendicular to the bony surface of the nasal bridge and may vary across the nasal bridge to maintain a perpendicular orientation to the nasal bridge.

Certain parameters of the heating mask 140 can be adjusted for optimal neurostimulation. For example, thickness can be adjusted to optimize the coupling between the external vibratory stimulus and the bony nasal bridge. Vibratory conductance of the material around the nasal bridge can be adjusted to optimize the transmission of external vibration resulting in bone conduction.

In alternate embodiments, the neurostimulation unit 510 includes an ultrasonic transducer (not shown) to transfer ultrasound directly to the sensory nerves (such as the anterior ethmoidal nerve in the nasal septum) to induce tear production reflex in the LFU. The ultrasonic transducer can be any ultrasonic transducer known in the art that is capable of neurostimulation and has a size suitable to be included as a component of the heating mask 140. The ultrasonic transducer can include an array of ultrasonic transducers that enable dispersed or focused ultrasound energy to maximally control the induced neuromodulation of the tear production reflex.

In alternate embodiments, the sequence and timing of the heating and vibration stimulation can be adjusted to obtain optimal tear film therapy. Specifically, the eyelid heating that reduces meibum viscosity may be applied first, while the resonant frequency stimulation vibration next mobilizes the meibum within the Meibomian Glands, followed by neurostimulation that, in addition to increasing mucin and aqueous production, induces expression of the mobilized meibum from the Meibomian Glands. This sequence, and length of time of each element of the sequence, as well as repetitions of all elements of the sequence, and total time of therapy, can all be adjusted to obtain an optimal therapeutic result.

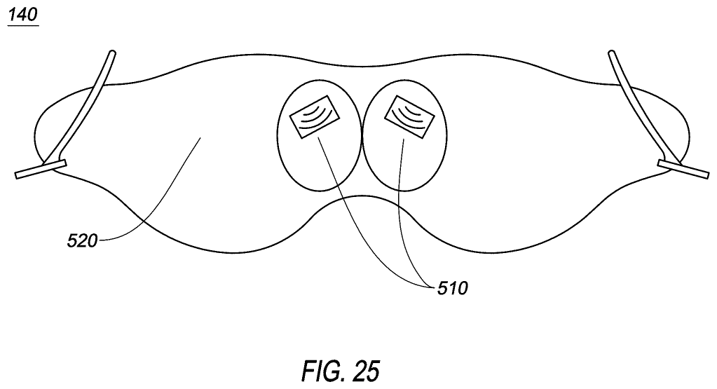

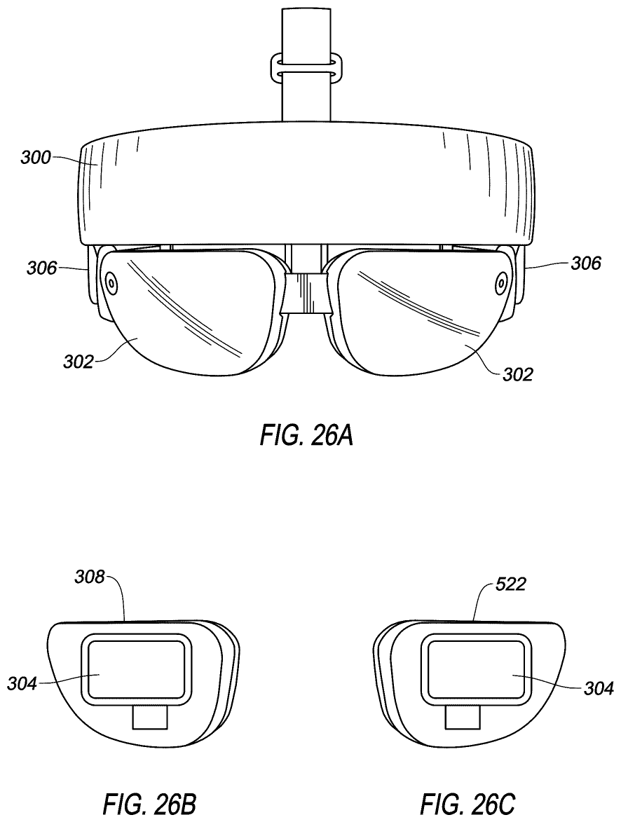

FIGS. 26A-26C show a moldable warming device incorporating a facial mapping device in each of the replaceable, adjustable, moldable eye pads of the mask. According to at least one embodiment, the mask 300 of the moldable warming device includes a pair of replaceable, adjustable, moldable, eye pads 302 including a facial mapping device. The facial mapping device allows the use of laser light and mathematical algorithms to determine raw height data information of the periocular surface of the user's eyelid. Transforming this data into topography data, a medical professional can use the topography data to calculate and map the contour of the ocular surface of the user's eyelid for independently adjusting the final location of each eye pads 302 before patient treatment. The real-time topography data allows the medical professional to determine the precise location for each of the eye pads 302 for optimizing the application of heat and harmonic resonance frequency vibration across the entire surface of the user's eyelid to ensure shear-thinning for improved Meibomian gland expression. The improved Meibomian gland expression through shear thinning results in improved Meibum lipid mobilization increasing the amount of Meibum fluid coating the aqueous layer of the eye for each blinking event.

According to an embodiment, as further shown in FIGS. 26B and 26C, each eye pad 302 includes a miniaturized version of the Optical Coherence Tomography (OCT) device 304. The OCT device 304 generates cross-sectional images with high axial resolution for tissue diagnosis. The OCT device 304 is useful in ophthalmology as the transparency of the ocular media allows for imaging of the user's retina even at the back of the eye. The OCT device 304 determines a frequency-domain measurement of spectral interference which allows for greater acquisition speed and greater contrast in the resulting images. The OCT device 304, according to embodiments of the invention, provides structural imaging of the periocular surface only. Using these methods, topography of the periocular surface is mapped, in real-time, including a mapping of the eyes and the nose including interpupillary distance and corneal apex height above lateral canthus. The precise location for positioning each the eye pads 302 can be calculated and will be used to quantify the position of the right and left eye pads 302 and automatically correlate the adjustment of both eye pads 302 together, so that both eye pads 302, in concert, expand or contract their lateral position initially based on interpupillary distance. This automated device is achieved through feedback controlled mechanical gearing setup inside the mask 300 responding to the topography data provided to the external computer-controlled software and algorithm locating the optimal position for the eye pads 302, depending on the patient's periocular topography. This adjustment can be achieved manually by the available manual adjustment wheels, right and left eye pad adjustment wheels 306, but relies on the data provided by the mapping module to guide the manual adjustment. According to an embodiment, the software can be configured to allow manual adjustment, but provides a visual rendering of the patient's periocular topography on an external screen responding to any misalignment with audible and visual feedback notifying the adjusting personnel of the potential approaching misalignment of the eye pads 302.

According to an embodiment, the real-time mapping of the periocular surface will also be used to quantify the position of the right and left eye pads 302 and automatically correlate the adjustment of both eye pads 302 together, so that both eye pads 302, in concert, expand or contract their lateral position based on the measurement of corneal apex relative to the lateral canthus. This automated module is achieved through feedback controlled mechanical gearing setup inside the mask 300 utilizing the topography data provided to the software by a replaceable topography mapping diagnostic pad 308 and the OCT device 304 and algorithms locating the optimal position for the eye pads 302, depending on the patient's periocular topography. This additional adjustment of the eye pads 302 is performed in concert with the initial adjustment of the eye pads 302 to optimize the location of the eye pads 302. This adjustment can be achieved manually by the available manual adjustment wheels, right and left eye pad adjustment wheels 306, but relies on the data provided by the mapping module to guide the manual adjustment. According to an embodiment, the software can be configured to allow manual adjustment, but provides a visual rendering of the patient's periocular topography on an external screen responding to any misalignment with audible and visual feedback notifying the adjusting personnel of the potential approaching misalignment of the eye pads 302.

As shown in FIG. 27, the mask 300 may include a plurality of manual adjustment wheels, right and left eye pad adjustment wheels 306, configured to move the eye pads 302 left or right, forward and backward, and up and down.

According to an embodiment, the real-time mapping of the eyes and periocular surface of the user's eyes will be used to automatically adjust the topography of the eye pads 302 to optimally conform to the periocular surface of the user's eyes. This automated module is achieved through feedback controlled mechanical gearing setup inside the mask 300, responding to the topography data provided to the software and algorithm of the OCT 304, thereby locating the optimal position for each the eye pads 302, based on the patient's periocular topography. The moldable and conforming properties of the eye pads 302 along with the real-time mapping module of the mask 300 allow the surface of the eye pads 302 to be formed into the correct geometry to conform to the specific topography of the periocular surface of the user's eyes strictly adhering to the pressure parameters detailed in this application.

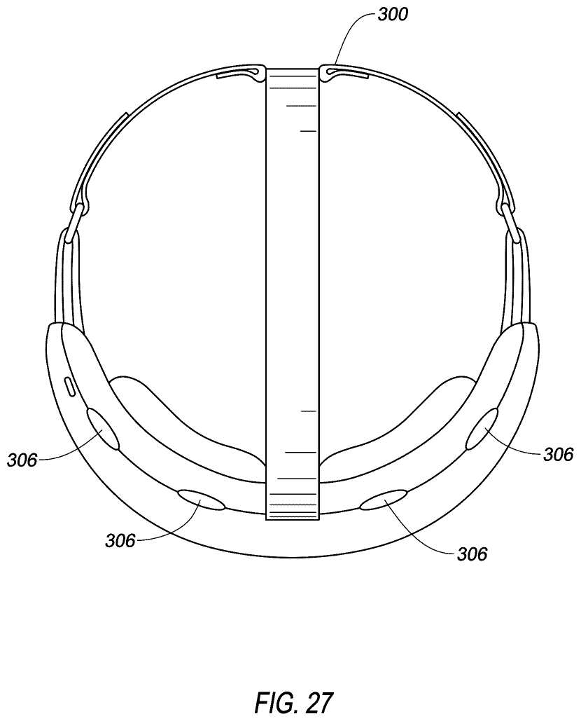

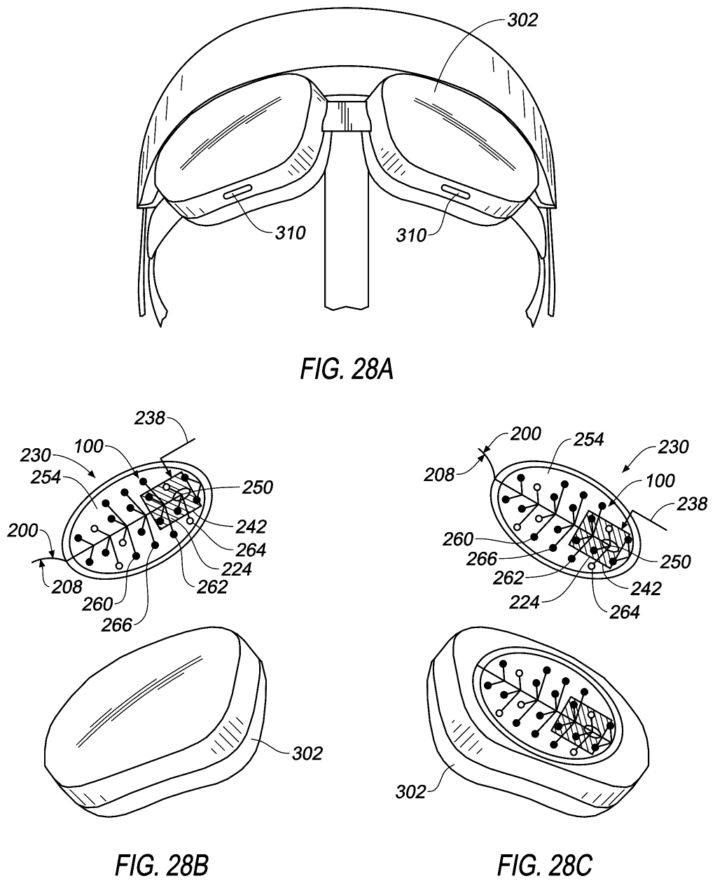

FIGS. 28A-28C show a moldable warming device incorporating a facial mapping module in each of the replaceable, adjustable, moldable eye pads of the mask. The mask 300, as shown in FIGS. 28A-28C, further includes a sensor array 230. The sensor array 230 can include piezoresistive, piezoelectric, optical, capacitive and/or elastoresistive sensors. A sensor array 230 is embedded in each of the eye pads 302 and can provide tactile feedback allowing precise automatic adjustment of the eye pads 302, 304 to the periocular surface of the user's eyes assuring optimal performance by the mask 300. The module is a snap into place device making its connections and structural support mate with the headset. The release mechanism is a button whereupon applied pressure from a human digit moves the “snap” or hook to the release position allowing the module and therefore the connections to the be able to be removed from the mask.

Embodiments provide the real-time mapping of the eyes and periocular surface will be used to manually adjust the eye pad 302 topographies to optimally conform to the periocular surface. This manual module is achieved through feedback controlled mechanical gearing setup inside the mask 300 responding to the topography data provided to the software and algorithm locating the optimal position for the eye-pads 302 depending on the patient's periocular topography. The moldable and conforming properties of the eye-pads 302 along with the mechanical features inside allow the surface of the eye-pads 302 to be formed into the correct geometry and conform to the specific topography of the periocular surface. The use of tactile sensors (e.g., 260, 262, 264, 266) in the sensor array can include piezoresistive, piezoelectric, optical, capacitive and or elastoresistive sensors. These embedded sensors 230 in the smart eye-pad 302 can produce tactile feedback allowing precise manual adjustment of the smart eye-pad 302 to the periocular surface assuring optimal performance by the mask 300. A manual status will be available to override the automated program allowing manual control with manual adjustment wheels 306 connected to the internal gearing of the smart eye-pad 302.

FIGS. 29A-29C show a moldable warming device incorporating a corneal fluorescein staining pad in each of the replaceable, adjustable, moldable eye pads of the mask. Corneal fluorescein staining is an important diagnostic test for treatment of dry eye disease (DED). (see R. Kourukmas, M. Roth, and G. Geerling Automated vs Human evaluation of corneal staining) However, the result of this test is dependent on the human grader. Embodiments of the invention include a corneal fluorescein staining pad 312 in each of the replaceable, adjustable, moldable eye pads 302 of the mask 300 including modified versions of the software-assisted grading algorithms developed by R Kourukmas, et. al. for improving the corneal fluorescein staining test. Each of the corneal fluorescein pads 312 provides 2 μl of 2% fluorescein injected onto the surface of the eyes through an injector 314. The algorithm for detecting and counting superficial keratitis using imaging is collected and automatically graded using a data base comparison of high experience ophthalmologists using Oxford graded scheme images for corneal staining at baseline and after 6-8 weeks. The software-assisted grading has consistent interrater agreement and implemented into the corneal fluorescein pads 312 is a consistent and reliable way to remove the inconsistency of different levels of years of experience from human grader to grader. The variance in human grading of corneal staining—if only small—is likely to have only little impact on clinical management and thus seems to be acceptable. While human graders provide results sufficient for clinical application, software-assisted grading of corneal staining ensures higher consistency and thus is preferable for re-evaluating patients, for example, in clinical trials. According to another embodiment, the corneal fluorescein pads 312 may be replaced with a corneal pad 312 with illumination of selectable variable wavelengths, for example, 465 nm to 490 nm may be used to illuminate the fluorescein dye, which fluoresces to wavelengths of 520 nm to 530 nm.

FIGS. 30A-30C show a moldable warming device incorporating a tear meniscus module in each of the replaceable, adjustable, moldable eye pads of the mask. According to an embodiment, each of the replaceable, adjustable, moldable eye pads 302 includes a tear interferometer 316 to measure tear meniscus. A patient normally has a tear meniscus height (TMH) of 0.2-0.5 mm, but in a patient with dry eye, it is usually less than 0.2 mm. The tear interferometer 316 embedded in each of the eye pads 302 for the mask 300 and modified existing software are used to assess TMH of the patient. It has been reported that the amounts of aqueous layer and lipid layer of the tear film compensate with each other for deficiencies to maintain homeostasis of the tear film. Thus, both these tear film components can be examined in the evaluation of DED. Applying tear interferometry to evaluate TMH has the advantage of noninvasive and automatic analyzing measurements of the tear film. (see Reiko Arita; Katsumi Yabusaki; Taisuke Hirono; Takanori Yamauchi; Tadashi Ichihashi; Shima Fukuoka; Naoyuki Morishige) The approach to measure TMH by tear interferometry is based on the interferometric reflection pattern and intensity histogram and is automated providing immediate feedback to the physician and patient. The measurement of interferometric TMH is thus highly repeatable based on the results and can be performed by trained nonphysician medical staff or with each of the eye pads 302.

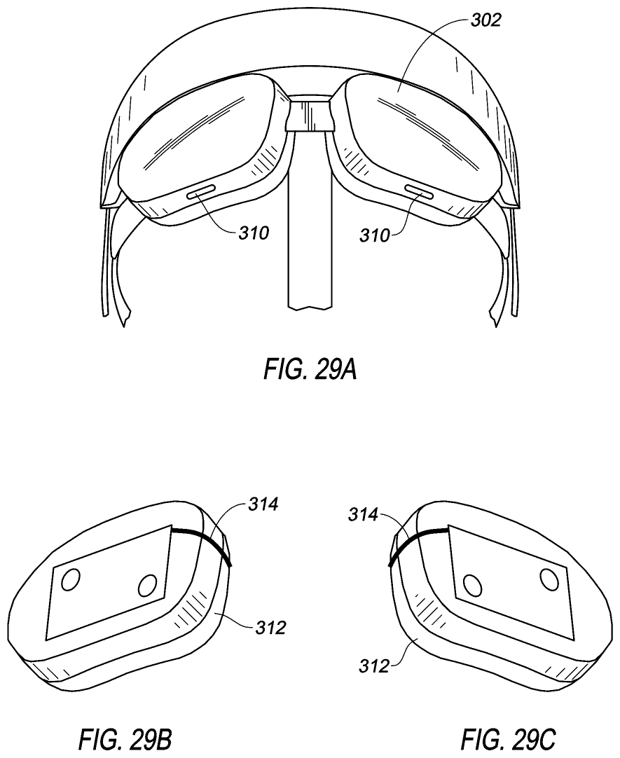

FIGS. 31A-31C show a moldable warming device having a conjunctival redness measurement module in each of the replaceable, adjustable, moldable eye pads of the mask. According to an embodiment, as shown in FIGS. 31B and 31C, conjunctival redness measurement modules 318 for the mask 300 are configured to diagnose and assess the level of conjunctival redness in the user's eyes. The conjunctiva, a thin, clear membrane that protects the eye, covering both the inside of the eyelid and the white of the eye (the sclera) in a continuous fashion. Goblet cells within the conjunctiva create the mucus layer that forms part of the tears. Conjunctival redness measurement modules 318 use embedded high speed digital miniature cameras 320 to send photographs to the observing physician who monitors and through modified software provides immediate feedback on the degree of dilation of conjunctival blood vessels in spherical conjunctiva into four levels: None, mild, moderate, and advanced. The software compares a developed, sophisticated database to the patient's photographs and provides the results in real-time.

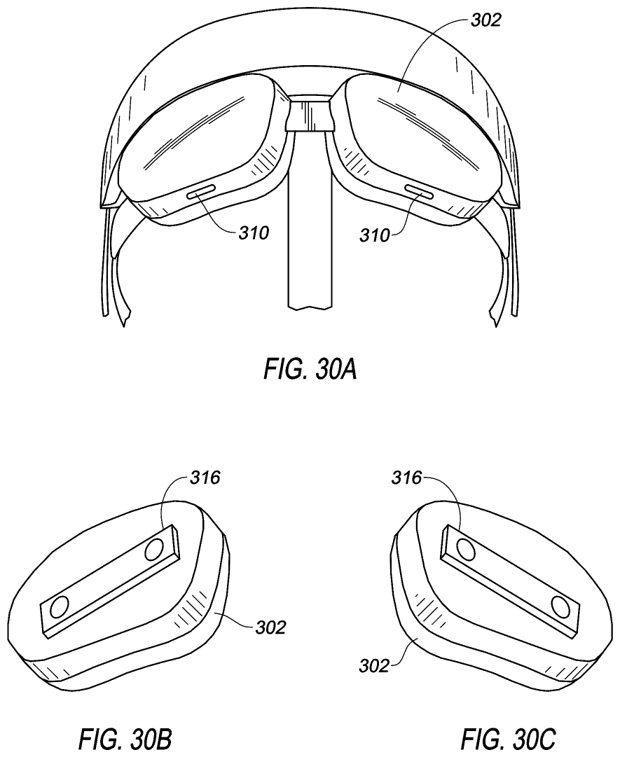

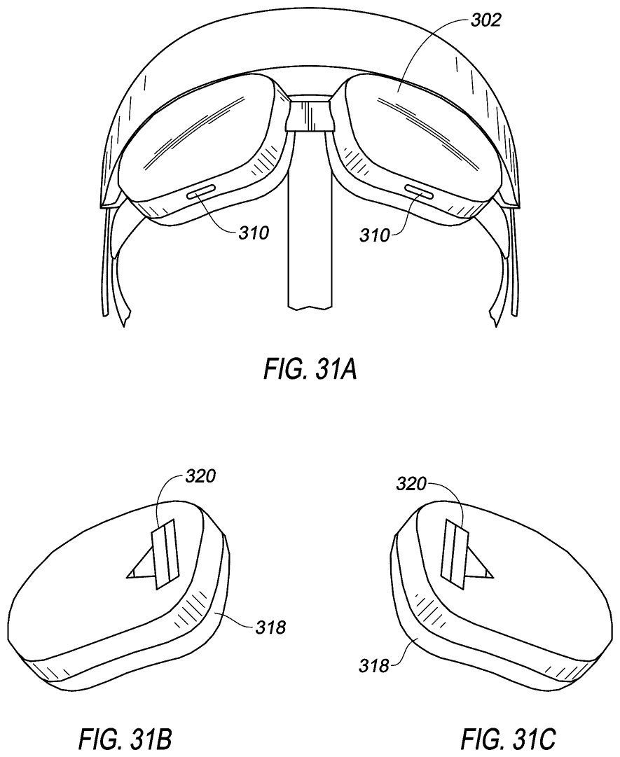

FIGS. 32A-32C show a moldable warming device incorporating a tear breakup time measurement module in each of the replaceable, adjustable, moldable eye pads of the mask. According to an embodiment, as shown in FIGS. 32B and 32C, tear breakup time measurement modules 322 for the mask 300 are configured to measure tear breakup time in both eyes. Tear film stability is measured automatically using an embedded high speed digital camera 324 in each of the eye pads 302 applying advanced software algorithms through detection of the break-up on the images reflected from the ocular surface in real-time. To assess the non-invasive tear film break-up time (NIBUT), the software assesses the different segments and distortion in the reflected mires. According to the National Eye Institute/Industry Workshop, tear film break-up time (TBUT) is accepted as the global criterion of dry eye, even though many studies criticized it as imprecise and unreproducible. In normal eyes, TBUT values range from 3 seconds (s) to 132 s, with an average of 27 s. A TBUT of less than 10 s suggests an abnormal tear film, with values of 5 s to 10 s considered marginal, and less than 5 s indicative of dry eye. The sensitivity and specificity of TBUT is 75% and 60%, respectively. The precise and immediate feedback provided by the tear breakup time measurement module 322 provides the physician and patient with accurate TBUT numbers to assist in the patients' examination.

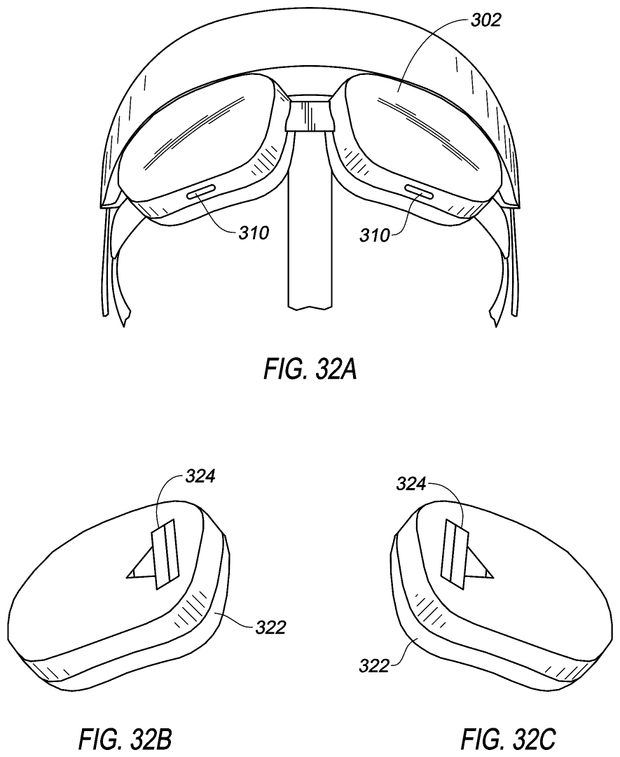

FIGS. 33A-33C show a moldable warming device incorporating a blink rate measurement module in each of the replaceable, adjustable, moldable eye pads of the mask. According to an embodiment, as shown in FIGS. 33B and 33C, blink rate measurement modules 326 for the mask 300 are configured to assess blink rate and completeness of blink cycle. Blink rate is measured in blinks/min and corresponds to the number of blinks occurring during a certain period. The blink duration is measured in seconds and corresponds to time difference between the start and end points of a blink. The spontaneous blink rate (SBR) plays a critical role in maintaining tear film stability and is crucial for sustaining tear film homeostasis. The blink accomplishes several tasks that are essential to preserving tear film homeostasis. During a complete blink, meibum is released from oil glands to the lid margin for dispersion onto the tear film. This is accomplished by the combined effort of the orbicularis oculi and the muscle of Riolan. The orbicularis compresses the tarsal plate to cause a milking effect on the meibomian glands, while the muscle of Riolan compresses the terminal ducts and acini of the glands. It is also believed that the muscle of Riolan keeps the meibomian glands closed to prevent leakage of meibum between blinks and during sleep. Each of the blink rate measurement modules 326 has a high-speed digital camera 328 taking bursts of film over a designated time, 3-5 minutes while the patient engages in SBR activity. The film is analyzed in real-time by advanced software able to count complete and partially complete patient blinks. This information is immediately available after the use of the blink rate measurement module 326 to the physician for patient examination and treatment. When blinks are detected with this algorithm, various measures can be performed and statistics over the recorded patient profiles can be monitored.

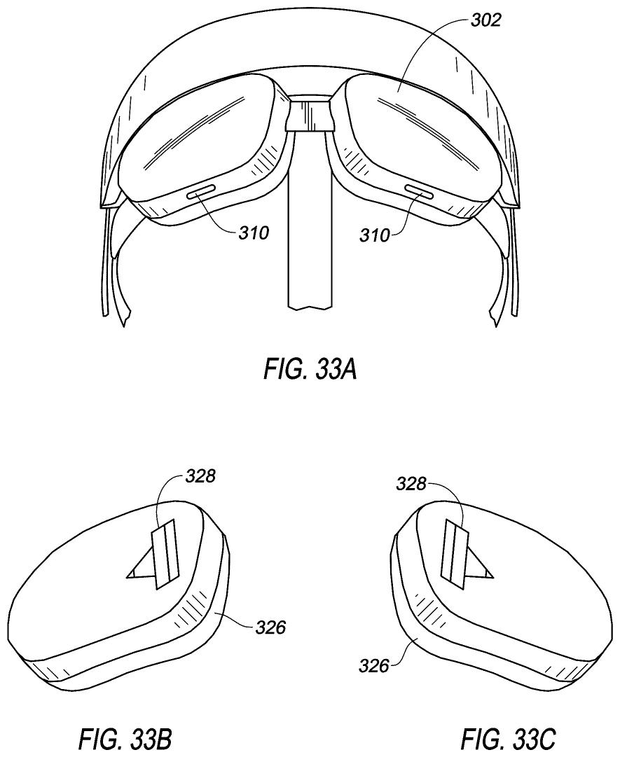

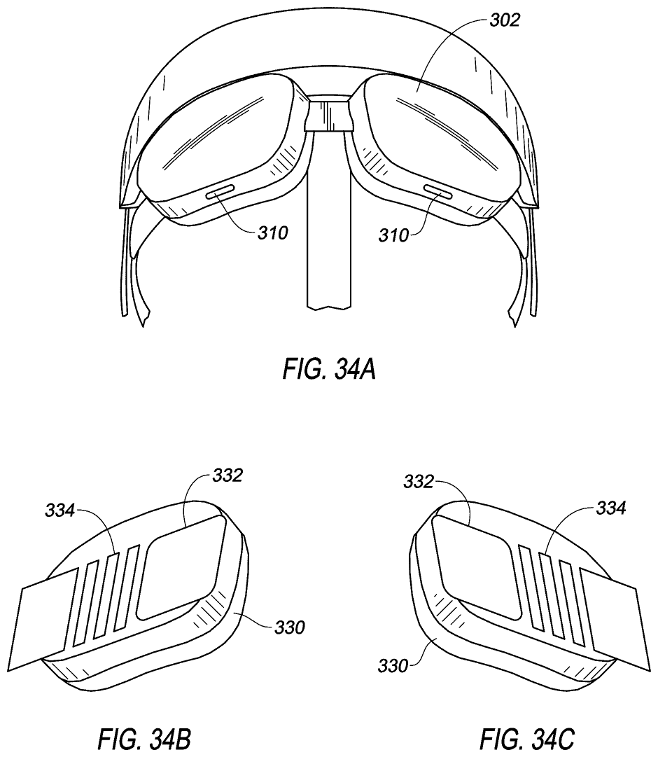

FIGS. 34A-34C show a moldable warming device incorporating an intraocular cell and flare measurement module in each of the replaceable, adjustable, moldable eye pads of the mask. According to an embodiment, as shown in FIGS. 34B and 34C, intraocular cell and flare measurement modules 330 for the mask 300 are configured to measure intraocular cell and flare in both eyes. Aqueous cells and flare are due to cellular infiltration and protein exudation into the anterior chamber. Aqueous cells are an early and definite sign of active inflammation. The translucence of the aqueous due to its high albumin content is called aqueous flare. A flare-up is an episode of worsening dry eye symptoms that occurs in response to an external trigger in patients who have dry eye disease. “Cell” is the individual inflammatory cells, while “flare” is the foggy appearance given by protein that has leaked from inflamed blood vessels. This finding is commonly seen with uveitis, iritis, and after surgery. However, when there are proteins present inside the fluid, the light may reflect in multiple directions, leading to the scattering of light. This is called the Tyndall effect and is due to the presence of turbidity in the aqueous humor. Each of intraocular cell and flare measurement modules 330 uses Laser Flare photometry (LFP) as the method of flare detection and quantitation under automated conditions. Its use allows an objective assessment of the degree of inflammation within the eye and is thus important in the management and follow-up of acute and chronic anterior uveitis. Each of the intraocular cell and flare measurement modules 330 incorporates a laser component 332 and photometry 334 to capture inflammation in the anterior chamber, thus transferring the images via advanced software to the physician's desktop providing immediate feedback from a broad database allowing the determination of the level of inflammation present in the patient. It also keeps this patient's data for correlation of present and past testing.

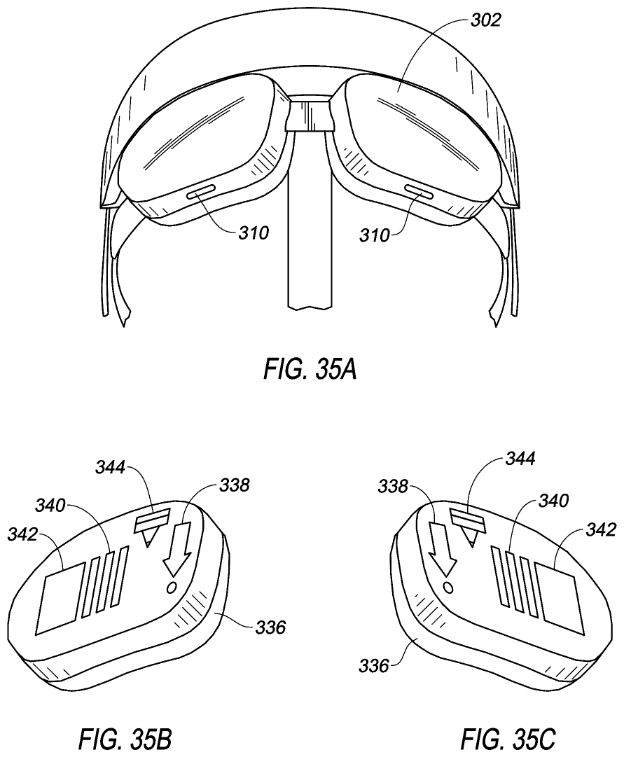

FIGS. 35A-35C show a moldable warming device incorporating an intraocular pressure measurement module in each of the replaceable, adjustable, moldable eye pads of the mask. According to an embodiment, as shown in FIGS. 35B and 35C, intraocular pressure measurement modules 336 for the mask 300 are configured to measure intraocular pressure (IOP). Goldmann applanation tonometers are currently considered the most reliable instruments for an accurate IOP measurement. These tonometers use the Imbert-Fick law: P=F/S, in which P is pressure, S represents the surface of the flattened area, and F is the force needed to flatten a fixed corneal area. Non-contact tonometry (NCT), also known as the “air puff test,” uses increasing air intensities to flatten the apex of the non-anesthetized cornea. The force used to flatten the cornea is detected by sensors, recorded, and converted to mm Hg. A brief puff of air is blown at the corneal surface of the eye. The patient will hear the puffing sound and feel a coolness or mild pressure on the eye. The tonometer records the IOP from the change in the light reflected off the cornea as it is indented by the air puff. Many types of devices have been proposed for IOP measurement, such as a Goldmann applanation tonometry (GAT), a NCT, an iCare rebound tonometer, and a dynamic contour tonometer, each of which has advantages and disadvantages. The GAT is widely regarded as the gold standard for IOP measurement due to its accuracy and excellent reproducibility, while the NCT is most widely used in outpatients and in ocular hypertension screening because of its non-invasive and convenient nature. The intraocular pressure measurement modules 336 for the mask 300 utilize a variation of the NCT to measure the IOP of the patient's eyes. Each of the intraocular pressure measurement modules 336 for the mask 300 contains the air puff gun 338x, which has variable short air pulse features of known pressure, duration, and quantity of pulsed air. The infrared light 340 is also incorporated into the intraocular pressure measurement modules 336, where the pressure dependent deformation is estimated from the time when the eye becomes flat, which is derived from the monitored reflection of an incident infrared light 342. A high-speed miniature camera 344 captures the complete motion of the eye directly and obtains more data during the pressure measurement. Combining the data collected using advanced analytical software to collect and analyze the data in real-time provides the physician with the IOP number immediately for its use in patient care and feedback.

According to an embodiment, each of the replaceable, adjustable, moldable eye pads of the mask may include a cold compress for inflammation reduction. According to another embodiment, each of the replaceable, adjustable, moldable eye pads of the mask may include a surface available to hold hydrogel sheets or moisturized cotton pads, the hydrogel sheets or the moisturized cotton pads containing either moisture alone or impregnated with anti-inflammatory agents including steroids or non-steroidal anti-inflammatories or agents expanding therapies including blepharitis, demodex, or eyelid wrinkles.

While preferred embodiments have been shown and described herein, it will be obvious to those skilled in the art that such embodiments are provided by way of example only. Numerous variations, changes, and substitutions will now occur to those skilled in the art without departing from the invention. Various alternatives to the embodiments described herein may be employed in practicing the invention. It is intended that any claims presented define the scope of the various embodiments and that methods and structures within the scope of these claims and their equivalents be covered thereby.

Embodiments described herein, therefore, are well adapted to carry out the objects and attain the ends and advantages mentioned, as well as others inherent therein. While a presently preferred embodiment has been given for purposes of disclosure, numerous changes exist in the details of procedures for accomplishing the desired results. These and other similar modifications will readily suggest themselves to those skilled in the art and are intended to be encompassed within the spirit of the various embodiments disclosed herein and the scope of the appended claims.

Although the various embodiments have been described in detail, various changes, substitutions, and alterations can be made hereupon without departing from the principle and scope of the various embodiments. Accordingly, the scope of the various embodiments should be determined by the following claims and their appropriate legal equivalents.

The singular forms “a,” “an,” and “the” include plural referents, unless the context clearly dictates otherwise.

Optional or optionally means that the subsequently described event or circumstances can or may not occur. The description includes instances where the event or circumstance occurs and instances where it does not occur.

Ranges can be expressed herein as from about one particular value, and/or to about another particular value. When such a range is expressed, it is to be understood that another embodiment is from the one particular value and/or to the other particular value, along with all combinations within said range.

As used herein and in the appended claims, the words “comprise,” “has,” and “include” and all grammatical variations thereof are each intended to have an open, non-limiting meaning that does not exclude additional elements or steps.

As used herein, terms such as “first” and “second” are arbitrarily assigned and are merely intended to differentiate between two or more components of an apparatus. It is to be understood that the words “first” and “second” serve no other purpose and are not part of the name or description of the component, nor do they necessarily define a relative location or position of the component. Furthermore, it is to be understood that the mere use of the term “first” and “second” does not require that there be any “third” component, although that possibility is contemplated under the scope of the various embodiments.

While the invention has been described in conjunction with specific embodiments thereof, it is evident that many alternatives, modifications, and variations will be apparent to those skilled in the art considering the foregoing description. Accordingly, it is intended to embrace all such alternatives, modifications, and variations as fall within the spirit and broad scope of the appended claims. The embodiments can suitably comprise, consist or consist essentially of the elements disclosed and can be practiced in the absence of an element not disclosed.

What is claimed is:

1. A moldable warming device, comprising:
a heating disc;
a harmonic resonance frequency stimulation vibration generator (RFSVG);
a coupling device;
a mask configured to hold the heating disc, the harmonic RFSVG, and the coupling device for use in parallel utility; and
a sensor configured to determine tuning parameters of a harmonic resonance frequency stimulation vibration and heating profile of a user's individual eyelid and periorbital three-dimensional anatomy and surface topography,
wherein the moldable warming device is configured to sequentially and alternately apply, using the heating disc, therapeutic warmth and, using the harmonic RFSVG, tuned harmonic resonance frequency stimulation vibration according to the determined tuning parameters across the contour of the entire surface of the user's eyelid and periorbital structures to induce expression of shear thinned and liquified meibum from each Meibomian gland in all four eyelids to improve the lipid layer of the surface of the user's eye tear film, and
wherein the RFSVG is configured to generate the tuned harmonic resonance frequency stimulation vibration comprising a superposition of two or more longitudinal vibrational waves across the contour of the entire surface of the user's eyelid and periorbital structures, wherein a primary axis of the first vibrational wave is in a superior-inferior direction parallel to the depth of the user's Meibomian glands, and a primary axis of the second vibrational wave is in a medial-lateral direction tangential to the depth of the user's Meibomian glands, to induce expression of the shear thinned and liquified meibum from each Meibomian gland in all four eyelids to improve the lipid layer of the surface of the user's eye tear film.

2. The moldable warming device of claim 1, further comprising:
a pair of removable and reusable eye pads, each eye pad comprising a miniaturized optical coherence tomography device configured to
map, in real-time, a topography of the user's periocular surface, including a mapping of the eyes and the nose including interpupillary distance and corneal apex height above the lateral canthus, and
based on the real-time mapping, determine a precise location for positioning each eye pad to quantify the position of each eye pad and to correlate the adjustment of the pair of eye pads together, so that both eye pads, in concert, expand or contract their lateral position initially based on the interpupillary distance.

3. The moldable warming device of claim 2, wherein the miniaturized optical coherence tomography device is further configured to quantify, based on the real-time mapping, the position for each eye pad, wherein each eye pad is configured in concert to move in and out in a lateral direction, based on a measurement of the corneal apex height relative to the lateral canthus.

4. The moldable warming device of claim 2, wherein the miniaturized optical coherence tomography device is further configured to determine, based on the real-time mapping, a surface topography and a periocular surface of the user's eyes to optimize conformance of the pair of eye pads to the periocular surface of the user's closed eyelids.

5. The moldable warming device of claim 2, further comprising:
a plurality of manual adjustment wheels, each manual adjustment wheel configured to produce tactile feedback to the user of the moldable warming device allowing the user to manually adjust each eye pad to optimize conformance of the pair of eye pads to the periocular surface of the user's eyelids.

6. The moldable device of claim 1, wherein each eye pad comprises a module insert configured to perform advanced diagnostic enabled imaging, the module insert comprising one of:
a corneal staining pad, the corneal staining pad being one of a corneal fluorescein staining pad or a corneal rose bengal staining pad with illumination of selectable variable waverlengths, the corneal staining pad configured to detect dry eye syndrome,
a tear interferometer configured to measure a tear meniscus in the user's eyes,
a conjunctival redness measurement pad configured to diagnose and assess a level of conjunctival redness in the user's eyes,
a tear breakup time measurement pad configured to measure tear breakup time in the user's eyes,
a blink rate measurement pad configured to assess blink rate and completeness of blink cycle of the user's eyes for maintaining tear film stability and for sustaining tear film homeostasis,
an intraocular cell and flare measurement pad configured to measure intraocular cell and flare in the user's eyes, and
an intraocular pressure measurement pad configured to measure intraocular pressure in the user's eyes.

7. The moldable warming device of claim 2, wherein the pair of eye pads are configured to provide cold compress for inflammation reduction.

8. The moldable warming device of claim 2, wherein the pair of eye pads further comprise a surface available to hold hydrogel sheets or moisturized cotton pads, the hydrogel sheets or the moisturized cotton pads containing either moisture alone or impregnated with anti-inflammatory agents including steroids or non-steroidal anti-inflammatories or agents expanding therapies including blepharitis, demodex, or eyelid wrinkles.

9. A method for ophthalmic eyelid therapy, the method comprising the steps of:
applying a moldable warming device to a user's individual eyelid and periorbital three-dimensional anatomy and surface topography, the moldable warming device comprising:
a heating disc;
a harmonic resonance frequency stimulation vibration generator (RFSVG);
a coupling device;
a mask, wherein the mask is configured to hold the heating disc, the harmonic RFSVG, and the coupling device for use in parallel utility; and
a sensor;
determining, via the sensor, tuning parameters of a harmonic resonance frequency stimulation vibration and heating profile of the user's individual eyelid and periorbital three-dimensional anatomy and surface topography;
sequentially and alternately applying, using the heating disc, thermal energy and, using the harmonic RFSVG, harmonic resonance frequency stimulation vibration according to the determined heating profile of the user's individual eyelid and periorbital three-dimensional anatomy and surface topography and the tuning parameters; and
transferring the thermal energy and the harmonic resonance frequency stimulation vibration across the contour of the entire surface of the user's eyelid and periorbital structures; and to induce expression of shear thinned and liquified meibum from each Meibomian gland in all four eyelids to improve the lipid layer of the surface of the user's eye tear film,
wherein the harmonic resonance frequency stimulation vibration comprises a superposition of two or more longitudinal vibrational waves across the contour of the entire surface of the user's eyelid and periorbital structures, wherein a primary axis of the first vibrational wave is in a superior-inferior direction parallel to the depth of the user's Meibomian glands, and a primary axis of the second vibrational wave is in a medial-lateral direction tangential to the depth of the user's Meibomian glands to induce expression of shear thinned and liquified meibum from each Meibomian gland in all four eyelids to improve the lipid layer of the surface of the user's eye tear film.

10. The method for ophthalmic eyelid therapy of claim 9, wherein each eye pad comprises a miniaturized optical coherence tomography device,
the method further comprising, using the miniaturized optical coherence tomography device,
mapping, in real-time, a topography of the user's periocular surface, including a mapping of the eyes and the nose including interpupillary distance and corneal apex height above the lateral canthus, and
based on the real-time mapping, determining a precise location for positioning each eye pad to quantify the position of each eye pad and to correlate the adjustment of the pair of eye pads together, so that both eye pads, in concert, expand or contract their lateral position initially based on the interpupillary distance.

11. The method for ophthalmic eyelid therapy of claim 10, further comprising:
quantifying, using the miniaturized optical coherence tomography device, based on the real-time mapping, the position for each eye pad, wherein each eye pad is configured in concert to move in and out in a lateral direction, based on a measurement of the corneal apex height relative to the lateral canthus.

12. The method for ophthalmic eyelid therapy of claim 10, further comprising:
determining, based on the real-time mapping, a surface topography and a periocular surface of the user's eyes to optimize conformance of the pair of eye pads to the periocular surface of the user's closed eyelids.

13. The method for ophthalmic eyelid therapy of claim 10, wherein the moldable warming device further comprises a plurality of manual adjustment wheels, the method further comprising:
manually adjusting, using each manual adjustment wheel configured to produce tactile feedback to the user of the moldable warming device, each eye pad to optimize conformance of the pair of eye pads to the periocular surface of the user's eyelids.

14. The method for ophthalmic eyelid therapy of claim 10, wherein each eye pad comprises a module insert, the method further comprising,
performing, using the module insert, advanced diagnostic enabled imaging,
wherein the performing step comprises:
detecting, using a corneal staining pad, the corneal staining pad being one of a corneal fluorescein staining pad or a corneal rose bengal staining pad with illumination of selectable variable waverlengths, dry eye syndrome,
measuring, using a tear interferometer, a tear meniscus in the user's eyes,
diagnosing and assessing, using a conjunctival redness measurement pad, a level of conjunctival redness in the user's eyes,
measuring, using a tear breakup time measurement pad, tear breakup time in the user's eyes,
assessing, using a blink rate measurement pad, blink rate and completeness of blink cycle of the user's eyes for maintaining tear film stability and for sustaining tear film homeostasis,
measuring, using an intraocular cell and flare measurement pad, intraocular cell and flare in the user's eyes, and
measuring, using an intraocular pressure measurement pad, intraocular pressure in the user's eyes.

15. The method for ophthalmic eyelid therapy of claim 10, wherein the pair of eye pads are configured to provide cold compress for inflammation reduction.

16. The method for ophthalmic eyelid therapy of claim 10, wherein the pair of eye pads further comprise a surface available to hold hydrogel sheets or moisturized cotton pads, the hydrogel sheets or the moisturized cotton pads containing either moisture alone or impregnated with anti-inflammatory agents including steroids or non-steroidal anti-inflammatories or agents expanding therapies including blepharitis, demodex, or eyelid wrinkles.
